(19) United States
(12) Patent Application Publication
(10) Pub. No.: US 2020/0395112 A1
(43) Pub. Date: Dec. 17, 2020

(54) A System and Method for Documenting a Patient Medical History

(71) Applicant: Cardio Holding BV, Delft (NL)

(72) Inventor: Eelko Ronner, Delft (NL)

(73) Assignee: Cardio Holding BV, Delft (NL)

(21) Appl. No.: 16/970,921

(22) PCT Filed: Feb. 18, 2019
(86) PCT No.: PCT/IB2019/000167

§ 371 Date: Aug. 18, 2020

Related U.S. Application Data
(60) Provisional application No. 62/631,851, filed on Feb. 18, 2018.
(60) Provisional application No. 62/644,433, filed on Mar. 17, 2018.

(51) Int. Cl. G16H 15/00; G16H 10/60; G16H 10/20; G16H 50/30

(52) U.S. Cl. CPC G16H 15/00; G16H 50/30; G16H 10/20; G16H 10/60

(57) ABSTRACT

A system and method for documenting a medical history of a patient including presenting a first graphical user interface having a graphics and text based selection for taking in medical history data, the graphics based selection includes a numeric pad superimposed on a human body; monitoring alarming information contained in said selection; presenting a second graphical user interface having icons depicting pain or symptom in certain areas of a body and a selection box for each of the depicted pain or symptom; tracking symptoms, not diseases; presenting a third graphical user interface having icons with corresponding response boxes configured to question and obtain responses regarding a medical health history; and presenting a fourth graphical user interface having icons depicting current health conditions and selection boxes for each of the depicted current health conditions to obtain at least one response regarding said current health condition.

### CROSS-REFERENCE TO RELATED APPLICATIONS

The present PCT patent application claims priority benefit of the U.S. provisional application for patent Ser. No. 62/631,851, entitled “Diagnostic Booth”, filed on Feb. 18, 2018 under 35 U.S.C. 119(e) and U.S. provisional application for patent Ser. No. 62/644,433, entitled “Agile Block Chain medical history application”, filed on Mar. 17, 2018 under 35 U.S.C. 119(e). The present Utility patent application is the National Phase filing under 35 U.S.C. 371 of the International Application No PCT/IB2019/000167 filed 18 Feb. 2019 entitled “A System and Method for Documenting a Patient Medical History”. The contents of these related provisional and PCT applications are incorporated herein by reference for all purposes to the extent that such subject matter is not inconsistent herewith or limiting hereof.

### INCORPORATION BY REFERENCE OF SEQUENCE LISTING PROVIDED AS ATEXT FILE

Not applicable.

### FEDERALLY SPONSORED RESEARCH OR DEVELOPMENT

Not applicable.

### REFERENCE TO SEQUENCE LISTING, A TABLE, OR A COMPUTER LISTING APPENDIX

Not applicable.

### COPYRIGHT NOTICE

A portion of the disclosure of this patent document contains material that is subject to copyright protection by the author thereof. The copyright owner has no objection to the facsimile reproduction by anyone of the patent document or patent disclosure for the purposes of referencing as patent prior art, as it appears in the Patent and Trademark Office, patent file or records, but otherwise reserves all copyright rights whatsoever.

### BACKGROUND OF THE RELEVANT PRIOR ART

One or more embodiments of the invention generally relate to medical data collection and analysis. More particularly, certain embodiments of the invention relate to systems and methods for improving quality of a patient's medical data and improving the quality of related data analysis.

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon. Medical history (so called anamnesis) is one starting point for a journey through health care. This might lead to over and/or under treatment if not performed well. Medical data and research on medical history taking is currently limited. Even more, statistics on medical history taken are largely unknown. For example, if specific complaints have an average self reported duration of an hour instead of 5 minutes does this influence the chance of having a certain disease? How much does this chance the likelihood quantitatively? And if this was reported by a man versus a woman? Do other diseases than also become more likely? Would other diagnostics be needed? What are the statistics? Is a rare but grave condition possible if more details in medical history are known? When should extra data be gathered? These largely unknown, not quantified aspects of medical history taking could perhaps contribute to better health and or better health care.

Given Artificial Intelligence (A.I.)/big data possibilities currently available medical history taking may be left behind in quantifiable analytic possibilities. Commonly there is only a doctor's transcript of a conversation mostly. There may be language or communication barriers, or lack of access to doctors or limited time. Medical history may be likely being shortened or jeopardized by these barriers. Time may be too short to fulfill all goals in a doctor physician consultation. Moreover, clearing up all specific details on medical complaints in a consultation may not be a patient's priority. It too leads to a lot of interruptions in conversation as described in research. Often an advice to wait and see may be given, as can be learned from the successful Dutch health care system where medical history taking works may be an effective goal keeper for more expensive care by general practitioners. The following is an example of a specific aspect in the prior art that, while expected to be helpful to further educate the reader as to additional aspects of the prior art, is not to be construed as limiting the present invention, or any embodiments thereof, to anything stated or implied therein or inferred thereupon. By way of educational background, another aspect of the prior art generally useful to be aware of is that in a doctor-patient interaction a visit may be a difficult undertaking for both parties. The physician or other health care worker may need to get a detailed grip on the health status of a patient by asking a large amount of structured questions to determine presence or absence of disease. A patient may experience this process completely different. A health care worker asking all these questions may not seem to listen, interrupts patient to get back on “medical history track”. And in the process the health care worker may keep on entering data but may fail to look at the patient as normal communication would require, where data entry is required. History taking by health care workers may be an extensive and required process, for example by use of the so-called “Socrates” acronym. This process may be completed with data on patient previous history, and co-morbidities and situations like smoking or drug abuse and social/psychological context.

Typically, medical history taking can be described as a scripted interview, where specific questions are a logical result of previous questions, for example, in case of a fever mentioned, common causes of infection are checked, like coughing, pain on micturition or infected wounds. These “scripts” are often based on logic and experience, working towards ruling in or ruling out disease states, more than the result of extensive (unbiased) scientific journeys. For many diseases, like heart disease in women, the determinants that rule in or rule out heart disease may not be clear. Even obvious aspects of a complaint, like the duration of a complaint, may not be linked to absence or presence of disease in a quantifiable fashion. Statistics are lacking to describe the likelihood that a specific pattern of answers fits with this or that disease. More over, the extensive patterns of complaints one might have, are often not elucidated by doctors, as certain facts do not turn up in the scripted interview that forms the anamnesis (=medical history). One could see this, as “leading the witness”, doctors may ask questions in a specific script, focusing on the patterns known. For example, in case of chest pain, a doctor may likely to ask for radiation of pain to the left arm, but not for possible radiating pain to the left leg. This conventional technique of anamnesis is focused on finding disease, our method challenges this by focusing on getting details of the complaint, and not missing other complaints. We focus on elucidating complaints, and the specifics to describe a complaint, any complaint, in general terms, without predetermined pathways or scripts towards specific diseases.

This too, can be adapted to, for example, a legal setting instead of our medical example here above. Our model in legal matters, aims to specify a legal issue by asking for any set of becoming specifics of a legal issue in an orderly fashion. Thus, again forming a quantified dataset. Again, a standard questioning structure is set up, (for example: who, what, where, when, why, how, how much, price, cost, cause, effect, goal, result) and each question rubricated to relevant categories as relevant in the setting. Adding to this structure, but not limited to, the index and or paragraphs of an extensive legal textbook and or jurisdiction regarding the specific matter is used in question form. In this setting, a legal issue is dissected by our questionnaire model in a large number of reproducible quantified bits, producing a usable dataset. This can be used as a starting point for a legal case to start of further information gathering, or to link to legal data of laws and or jurisdiction.

The database output part can be enhanced with legal answers to comparable cases and or laws and jurisdiction close to or related to the specific set of answers to the structured interview. Again, we do not work to conclusions, but aim to get the whole story in a “structured way” striving to get it in a quantified fashion for comparisons.

In view of the foregoing, it is clear that these traditional techniques are not perfect and leave room for more optimal approaches.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 illustrates an exemplary interface for taking medical history, in accordance with an embodiment of the present invention;

FIG. 2 illustrates exemplary use of icons during interfacing with a patient demonstrating situations in which symptoms occur, in accordance with an embodiment of the present invention;

FIGS. 3A & 3B illustrate exemplary questionnaire interfaces, where FIG. 3A illustrates use of icons for questioning a user's health history, where FIG. 3B illustrates use of icons for questioning a user's current health, in accordance with an embodiment of the invention;

FIG. 4 is an illustration of an overall data processing from an interfacing device to eventual data analysis, in accordance with an embodiment of the present invention;

FIG. 5 is an illustration of an exemplary paper-based questionnaire, in accordance with an embodiment of the present invention;

FIG. 6 illustrates a diagnostic booth, in accordance with an embodiment of the invention;

FIG. 7 illustrates an interface providing a presentation of medical history analysis results, in accordance with an embodiment of the invention;

FIG. 8 is a flow chart illustrating a method for performing data analytics on a medical history database, in accordance with en embodiment of the invention;

FIG. 9 illustrates a backbone model, in accordance with an embodiment of the present invention;

FIG. 10 is a block diagram illustrating a software system modules diagram, in accordance with an embodiment of the invention;

FIG. 11 is a block diagram depicting an exemplary client/server system which may be used by an exemplary web-enabled/networked embodiment of the present invention;

FIG. 12 illustrates a block diagram depicting a conventional client/server communication system, which may be used by an exemplary web-enabled/networked embodiment of the present invention.

Unless otherwise indicated illustrations in the figures are not necessarily drawn to scale.

### DETAILED DESCRIPTION OF SOME EMBODIMENTS

The present invention is best understood by reference to the detailed figures and description set forth herein.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

All words of approximation as used in the present disclosure and claims should be construed to mean “approximate,” rather than “perfect,” and may accordingly be employed as a meaningful modifier to any other word, specified parameter, quantity, quality, or concept. Words of approximation, include, yet are not limited to terms such as “substantial”, “nearly”, “almost”, “about”, “generally”, “largely”, “essentially”, “closely approximate”, etc.

As will be established in some detail below, it is well settled law, as early as 1939, that words of approximation are not indefinite in the claims even when such limits are not defined or specified in the specification.

For example, see Ex parte Mallory, 52 USPQ 297, 297 (Pat. Off. Bd. App. 1941) where the court said “The examiner has held that most of the claims are inaccurate because apparently the laminar film will not be entirely eliminated. The claims specify that the film is “substantially” eliminated and for the intended purpose, it is believed that the slight portion of the film which may remain is negligible. We are of the view, therefore, that the claims may be regarded as sufficiently accurate.”

Note that claims need only “reasonably apprise those skilled in the art” as to their scope to satisfy the definiteness requirement. See Energy Absorption Sys., Inc. v. Roadway Safety Servs., Inc., Civ. App. 96-1264, slip op. at 10 (Fed. Cir. Jul. 3, 1997) (unpublished) Hybridtech v. Monoclonal Antibodies, Inc., 802 F.2d 1367, 1385, 231 USPQ 81, 94 (Fed. Cir. 1986), cert. denied, 480 U.S. 947 (1987). In addition, the use of modifiers in the claim, like “generally” and “substantial,” does not by itself render the claims indefinite. See Seattle Box Co. v. Industrial Crating & Packing, Inc., 731 F.2d 818, 828-29, 221 USPQ 568, 575-76 (Fed. Cir. 1984).

Moreover, the ordinary and customary meaning of terms like “substantially” includes “reasonably close to: nearly, almost, about”, connoting a term of approximation. See In re Frye, Appeal No. 2009-006013, 94 USPQ2d 1072, 1077, 2010 WL 889747 (B.P.A.I. 2010) Depending on its usage, the word “substantially” can denote either language of approximation or language of magnitude. Deering Precision Instruments, L.L.C. v. Vector Distribution Sys., Inc., 347 F.3d 1314, 1323 (Fed. Cir. 2003) (recognizing the “dual ordinary meaning of th[e] term [“substantially”] as connoting a term of approximation or a term of magnitude”). Here, when referring to the “substantially halfway” limitation, the Specification uses the word “approximately” as a substitute for the word “substantially” (Fact 4). (Fact 4). The ordinary meaning of “substantially halfway” is thus reasonably close to or nearly at the midpoint between the forwardmost point of the upper or outsole and the rearwardmost point of the upper or outsole.

Similarly, the term ‘substantially’ is well recognized in case law to have the dual ordinary meaning of connoting a term of approximation or a term of magnitude. See Dana Corp. v. American Axle & Manufacturing, Inc., Civ. App. 04-1116, 2004 U.S. App. LEXIS 18265, *13-14 (Fed. Cir. Aug. 27, 2004) (unpublished). The term “substantially” is commonly used by claim drafters to indicate approximation. See Cordis Corp. v. Medtronic AVE Inc., 339 F.3d 1352, 1360 (Fed. Cir. 2003) (“The patents do not set out any numerical standard by which to determine whether the thickness of the wall surface is ‘substantially uniform.’ The term ‘substantially,’ as used in this context, denotes approximation. Thus, the walls must be of largely or approximately uniform thickness.”); see also Deering Precision Instruments, LLC v. Vector Distribution Sys., Inc., 347 F.3d 1314, 1322 (Fed. Cir. 2003); Epcon Gas Sys., Inc. v. Bauer Compressors, Inc., 279 F.3d 1022, 1031 (Fed. Cir. 2002). We find that the term “substantially” was used in just such a manner in the claims of the patents-in-suit: “substantially uniform wall thickness” denotes a wall thickness with approximate uniformity.

It should also be noted that such words of approximation as contemplated in the foregoing clearly limits the scope of claims such as saying ‘generally parallel’ such that the adverb ‘generally’ does not broaden the meaning of parallel. Accordingly, it is well settled that such words of approximation as contemplated in the foregoing (e.g., like the phrase ‘generally parallel’) envisions some amount of deviation from perfection (e.g., not exactly parallel), and that such words of approximation as contemplated in the foregoing are descriptive terms commonly used in patent claims to avoid a strict numerical boundary to the specified parameter. To the extent that the plain language of the claims relying on such words of approximation as contemplated in the foregoing are clear and uncontradicted by anything in the written description herein or the figures thereof, it is improper to rely upon the present written description, the figures, or the prosecution history to add limitations to any of the claim of the present invention with respect to such words of approximation as contemplated in the foregoing. That is, under such circumstances, relying on the written description and prosecution history to reject the ordinary and customary meanings of the words themselves is impermissible. See, for example, Liquid Dynamics Corp. v. Vaughan Co., 355 F.3d 1361, 69 USPQ2d 1595, 1600-01 (Fed. Cir. 2004). The plain language of phrase 2 requires a “substantial helical flow.” The term “substantial” is a meaningful modifier implying “approximate,” rather than “perfect.” In Cordis Corp. v. Medtronic AVE, Inc., 339 F.3d 1352, 1361 (Fed. Cir. 2003), the district court imposed a precise numeric constraint on the term “substantially uniform thickness.” We noted that the proper interpretation of this term was “of largely or approximately uniform thickness” unless something in the prosecution history imposed the “clear and unmistakable disclaimer” needed for narrowing beyond this simple-language interpretation. Id. In Anchor Wall Systems v. Rockwood Retaining Walls, Inc., 340 F.3d 1298, 1311 (Fed. Cir. 2003)” Id. at 1311. Similarly, the plain language of claim 1 requires neither a perfectly helical flow nor a flow that returns precisely to the center after one rotation (a limitation that arises only as a logical consequence of requiring a perfectly helical flow).

The reader should appreciate that case law generally recognizes a dual ordinary meaning of such words of approximation, as contemplated in the foregoing, as connoting a term of approximation or a term of magnitude; e.g., see Deering Precision Instruments, L.L.C. v. Vector Distrib. Sys., Inc., 347 F.3d 1314, 68 USPQ2d 1716, 1721 (Fed. Cir. 2003), cert. denied, 124 S. Ct. 1426 (2004) where the court was asked to construe the meaning of the term “substantially” in a patent claim. Also see Epcon, 279 F.3d at 1031 (“The phrase ‘substantially constant’ denotes language of approximation, while the phrase ‘substantially below’ signifies language of magnitude, i.e., not insubstantial.”). Also, see, e.g., Epcon Gas Sys., Inc. v. Bauer Compressors, Inc., 279 F.3d 1022 (Fed. Cir. 2002) (construing the terms “substantially constant” and “substantially below”); Zodiac Pool Care, Inc. v. Hoffinger Indus., Inc., 206 F.3d 1408 (Fed. Cir. 2000) (construing the term “substantially inward”); York Prods., Inc. v. Cent. Tractor Farm & Family Ctr., 99 F.3d 1568 (Fed. Cir. 1996) (construing the term “substantially the entire height thereof”); Tex. Instruments Inc. v. Cypress Semiconductor Corp., 90 F.3d 1558 (Fed. Cir. 1996) (construing the term “substantially in the common plane”). In conducting their analysis, the court instructed to begin with the ordinary meaning of the claim terms to one of ordinary skill in the art. Prima Tek, 318 F.3d at 1148. Reference to dictionaries and our cases indicates that the term “substantially” has numerous ordinary meanings. As the district court stated, “substantially” can mean “significantly” or “considerably.” The term “substantially” can also mean “largely” or “essentially.” Webster's New 20th Century Dictionary 1817 (1983).

Words of approximation, as contemplated in the foregoing, may also be used in phrases establishing approximate ranges or limits, where the end points are inclusive and approximate, not perfect; e.g., see AK Steel Corp. v. Sollac, 344 F.3d 1234, 68 USPQ2d 1280, 1285 (Fed. Cir. 2003) where it where the court said [W]e conclude that the ordinary meaning of the phrase “up to about 10%” includes the “about 10%” endpoint. As pointed out by AK Steel, when an object of the preposition “up to” is nonnumeric, the most natural meaning is to exclude the object (e.g., painting the wall up to the door). On the other hand, as pointed out by Sollac, when the object is a numerical limit, the normal meaning is to include that upper numerical limit (e.g., counting up to ten, seating capacity for up to seven passengers). Because we have here a numerical limit—“about 10%”—the ordinary meaning is that that endpoint is included.

In the present specification and claims, a goal of employment of such words of approximation, as contemplated in the foregoing, is to avoid a strict numerical boundary to the modified specified parameter, as sanctioned by Pall Corp. v. Micron Separations, Inc., 66 F.3d 1211, 1217, 36 USPQ2d 1225, 1229 (Fed. Cir. 1995) where it states “It is well established that when the term “substantially” serves reasonably to describe the subject matter so that its scope would be understood by persons in the field of the invention, and to distinguish the claimed subject matter from the prior art, it is not indefinite.” Likewise see Verve LLC v. Crane Cams Inc., 311 F.3d 1116, 65 USPQ2d 1051, 1054 (Fed. Cir. 2002). Expressions such as “substantially” are used in patent documents when warranted by the nature of the invention, in order to accommodate the minor variations that may be appropriate to secure the invention. Such usage may well satisfy the charge to “particularly point out and distinctly claim” the invention, 35 U.S.C. § 112, and indeed may be necessary in order to provide the inventor with the benefit of his invention. In Andrew Corp. v. Gabriel Elecs. Inc., 847 F.2d 819, 821-22, 6 USPQ2d 2010, 2013 (Fed. Cir. 1988) the court explained that usages such as “substantially equal” and “closely approximate” may serve to describe the invention with precision appropriate to the technology and without intruding on the prior art. The court again explained in Ecolab Inc. v. Envirochem, Inc., 264 F.3d 1358, 1367, 60 USPQ2d 1173, 1179 (Fed. Cir. 2001) that “like the term ‘about,’ the term ‘substantially’ is a descriptive term commonly used in patent claims to ‘avoid a strict numerical boundary to the specified parameter, see Ecolab Inc. v. Envirochem Inc., 264 F.3d 1358, 60 USPQ2d 1173, 1179 (Fed. Cir. 2001) where the court found that the use of the term “substantially” to modify the term “uniform” does not render this phrase so unclear such that there is no means by which to ascertain the claim scope.

Similarly, other courts have noted that like the term “about,” the term “substantially” is a descriptive term commonly used in patent claims to “avoid a strict numerical boundary to the specified parameter.”; e.g., see Pall Corp. v. Micron Seps., 66 F.3d 1211, 1217, 36 USPQ2d 1225, 1229 (Fed. Cir. 1995); see, e.g., Andrew Corp. v. Gabriel Elecs. Inc., 847 F.2d 819, 821-22, 6 USPQ2d 2010, 2013 (Fed. Cir. 1988) (noting that terms such as “approach each other,” “close to,” “substantially equal,” and “closely approximate” are ubiquitously used in patent claims and that such usages, when serving reasonably to describe the claimed subject matter to those of skill in the field of the invention, and to distinguish the claimed subject matter from the prior art, have been accepted in patent examination and upheld by the courts). In this case, “substantially” avoids the strict 100% nonuniformity boundary.

Indeed, the foregoing sanctioning of such words of approximation, as contemplated in the foregoing, has been established as early as 1939, see Ex parte Mallory, 52 USPQ 297, 297 (Pat. Off. Bd. App. 1941) where, for example, the court said “the claims specify that the film is “substantially” eliminated and for the intended purpose, it is believed that the slight portion of the film which may remain is negligible. We are of the view, therefore, that the claims may be regarded as sufficiently accurate.” Similarly, In re Hutchison, 104 F.2d 829, 42 USPQ 90, 93 (C.C.P.A. 1939) the court said “It is realized that “substantial distance” is a relative and somewhat indefinite term, or phrase, but terms and phrases of this character are not uncommon in patents in cases where, according to the art involved, the meaning can be determined with reasonable clearness.”

Hence, for at least the forgoing reason, Applicants submit that it is improper for any examiner to hold as indefinite any claims of the present patent that employ any words of approximation.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will be described in detail below with reference to embodiments thereof as illustrated in the accompanying drawings.

References to a “device,” an “apparatus,” a “system,” etc., in the preamble of a claim should be construed broadly to mean “any structure meeting the claim terms” exempt for any specific structure(s)/type(s) that has/(have) been explicitly disavowed or excluded or admitted/implied as prior art in the present specification or incapable of enabling an object/aspect/goal of the invention. Furthermore, where the present specification discloses an object, aspect, function, goal, result, or advantage of the invention that a specific prior art structure and/or method step is similarly capable of performing yet in a very different way, the present invention disclosure is intended to and shall also implicitly include and cover additional corresponding alternative embodiments that are otherwise identical to that explicitly disclosed except that they exclude such prior art structure(s)/step(s), and shall accordingly be deemed as providing sufficient disclosure to support a corresponding negative limitation in a claim claiming such alternative embodiment(s), which exclude such very different prior art structure(s)/step(s) way(s).

From reading the present disclosure, other variations and modifications will be apparent to persons skilled in the art. Such variations and modifications may involve equivalent and other features which are already known in the art, and which may be used instead of or in addition to features already described herein.

Although Claims have been formulated in this Application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any Claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable subcombination. The Applicants hereby give notice that new Claims may be formulated to such features and/or combinations of such features during the prosecution of the present Application or of any further Application derived therefrom.

References to “one embodiment,” “an embodiment,” “example embodiment,” “various embodiments,” “some embodiments,” “embodiments of the invention,” etc., may indicate that the embodiment(s) of the invention so described may include a particular feature, structure, or characteristic, but not every possible embodiment of the invention necessarily includes the particular feature, structure, or characteristic. Further, repeated use of the phrase “in one embodiment,” or “in an exemplary embodiment,” “an embodiment,” do not necessarily refer to the same embodiment, although they may. Moreover, any use of phrases like “embodiments” in connection with “the invention” are never meant to characterize that all embodiments of the invention must include the particular feature, structure, or characteristic, and should instead be understood to mean “at least some embodiments of the invention” include the stated particular feature, structure, or characteristic.

References to “user”, or any similar term, as used herein, may mean a human or non-human user thereof. Moreover, “user”, or any similar term, as used herein, unless expressly stipulated otherwise, is contemplated to mean users at any stage of the usage process, to include, without limitation, direct user(s), intermediate user(s), indirect user(s), and end user(s). The meaning of “user”, or any similar term, as used herein, should not be otherwise inferred or induced by any pattern(s) of description, embodiments, examples, or referenced prior-art that may (or may not) be provided in the present patent.

References to “end user”, or any similar term, as used herein, is generally intended to mean late stage user(s) as opposed to early stage user(s). Hence, it is contemplated that there may be a multiplicity of different types of “end user” near the end stage of the usage process. Where applicable, especially with respect to distribution channels of embodiments of the invention comprising consumed retail products/services thereof (as opposed to sellers/vendors or Original Equipment Manufacturers), examples of an “end user” may include, without limitation, a “consumer”, “buyer”, “customer”, “purchaser”, “shopper”, “enjoyer”, “viewer”, or individual person or non-human thing benefiting in any way, directly or indirectly, from use of. or interaction, with some aspect of the present invention.

In some situations, some embodiments of the present invention may provide beneficial usage to more than one stage or type of usage in the foregoing usage process. In such cases where multiple embodiments targeting various stages of the usage process are described, references to “end user”, or any similar term, as used therein, are generally intended to not include the user that is the furthest removed, in the foregoing usage process, from the final user therein of an embodiment of the present invention.

Where applicable, especially with respect to retail distribution channels of embodiments of the invention, intermediate user(s) may include, without limitation, any individual person or non-human thing benefiting in any way, directly or indirectly, from use of, or interaction with, some aspect of the present invention with respect to selling, vending, Original Equipment Manufacturing, marketing, merchandising, distributing, service providing, and the like thereof.

References to “person”, “individual”, “human”, “a party”, “animal”, “creature”, or any similar term, as used herein, even if the context or particular embodiment implies living user, maker, or participant, it should be understood that such characterizations are sole by way of example, and not limitation, in that it is contemplated that any such usage, making, or participation by a living entity in connection with making, using, and/or participating, in any way, with embodiments of the present invention may be substituted by such similar performed by a suitably configured non-living entity, to include, without limitation, automated machines, robots, humanoids, computational systems, information processing systems, artificially intelligent systems, and the like. It is further contemplated that those skilled in the art will readily recognize the practical situations where such living makers, users, and/or participants with embodiments of the present invention may be in whole, or in part, replaced with such non-living makers, users, and/or participants with embodiments of the present invention. Likewise, when those skilled in the art identify such practical situations where such living makers, users, and/or participants with embodiments of the present invention may be in whole, or in part, replaced with such non-living makers, it will be readily apparent in light of the teachings of the present invention how to adapt the described embodiments to be suitable for such non-living makers, users, and/or participants with embodiments of the present invention. Thus, the invention is thus to also cover all such modifications, equivalents, and alternatives falling within the spirit and scope of such adaptations and modifications, at least in part, for such non-living entities.

Headings provided herein are for convenience and are not to be taken as limiting the disclosure in any way.

The enumerated listing of items does not imply that any or all of the items are mutually exclusive, unless expressly specified otherwise.

It is understood that the use of specific component, device and/or parameter names are for example only and not meant to imply any limitations on the invention. The invention may thus be implemented with different nomenclature/terminology utilized to describe the mechanisms/units/structures/components/devices/parameters herein, without limitation. Each term utilized herein is to be given its broadest interpretation given the context in which that term is utilized.

Terminology. The following paragraphs provide definitions and/or context for terms found in this disclosure (including the appended claims):

“Comprising” And “contain” and variations of them—Such terms are open-ended and mean “including but not limited to”. When employed in the appended claims, this term does not foreclose additional structure or steps. Consider a claim that recites: “A memory controller comprising a system cache . . . ” Such a claim does not foreclose the memory controller from including additional components (e.g., a memory channel unit, a switch).

“Configured To.” Various units, circuits, or other components may be described or claimed as “configured to” perform a task or tasks. In such contexts, “configured to” or “operable for” is used to connote structure by indicating that the mechanisms/units/circuits/components include structure (e.g., circuitry and/or mechanisms) that performs the task or tasks during operation. As such, the mechanisms/unit/circuit/component can be said to be configured to (or be operable) for perform(ing) the task even when the specified mechanisms/unit/circuit/component is not currently operational (e.g., is not on). The mechanisms/units/circuits/components used with the “configured to” or “operable for” language include hardware—for example, mechanisms, structures, electronics, circuits, memory storing program instructions executable to implement the operation, etc. Reciting that a mechanism/unit/circuit/component is “configured to” or “operable for” perform(ing) one or more tasks is expressly intended not to invoke 35 U.S.C. sctn. 112, sixth paragraph, for that mechanism/unit/circuit/component. “Configured to” may also include adapting a manufacturing process to fabricate devices or components that are adapted to implement or perform one or more tasks.

“Based On.” As used herein, this term is used to describe one or more factors that affect a determination. This term does not foreclose additional factors that may affect a determination. That is, a determination may be solely based on those factors or based, at least in part, on those factors. Consider the phrase “determine A based on B.” While B may be a factor that affects the determination of A, such a phrase does not foreclose the determination of A from also being based on C. In other instances, A may be determined based solely on B.

The terms “a”, “an” and “the” mean “one or more”, unless expressly specified otherwise.

All terms of exemplary language (e.g., including, without limitation, “such as”, “like”, “for example”, “for instance”, “similar to”, etc.) are not exclusive of any other, potentially, unrelated, types of examples; thus, implicitly mean “by way of example, and not limitation . . . ”, unless expressly specified otherwise.

Unless otherwise indicated, all numbers expressing conditions, concentrations, dimensions, and so forth used in the specification and claims are to be understood as being modified in all instances by the term “about.” Accordingly, unless indicated to the contrary, the numerical parameters set forth in the following specification and attached claims are approximations that may vary depending at least upon a specific analytical technique.

The term “comprising,” which is synonymous with “including,” “containing,” or “characterized by” is inclusive or open-ended and does not exclude additional, unrecited elements or method steps. “Comprising” is a term of art used in claim language which means that the named claim elements are essential, but other claim elements may be added and still form a construct within the scope of the claim.

As used herein, the phase “consisting of” excludes any element, step, or ingredient not specified in the claim. When the phrase “consists of” (or variations thereof) appears in a clause of the body of a claim, rather than immediately following the preamble, it limits only the element set forth in that clause; other elements are not excluded from the claim as a whole. As used herein, the phase “consisting essentially of” and “consisting of” limits the scope of a claim to the specified elements or method steps, plus those that do not materially affect the basis and novel characteristic(s) of the claimed subject matter (see Norian Corp. v Stryker Corp., 363 F.3d 1321, 1331-32, 70 USPQ2d 1508, Fed. Cir. 2004). Moreover, for any claim of the present invention which claims an embodiment “consisting essentially of” or “consisting of” a certain set of elements of any herein described embodiment it shall be understood as obvious by those skilled in the art that the present invention also covers all possible varying scope variants of any described embodiment(s) that are each exclusively (i.e., “consisting essentially of”) functional subsets or functional combination thereof such that each of these plurality of exclusive varying scope variants each consists essentially of any functional subset(s) and/or functional combination(s) of any set of elements of any described embodiment(s) to the exclusion of any others not set forth therein. That is, it is contemplated that it will be obvious to those skilled how to create a multiplicity of alternate embodiments of the present invention that simply consisting essentially of a certain functional combination of elements of any described embodiment(s) to the exclusion of any others not set forth therein, and the invention thus covers all such exclusive embodiments as if they were each described herein.

With respect to the terms “comprising,” “consisting of,” and “consisting essentially of,” where one of these three terms is used herein, the disclosed and claimed subject matter may include the use of either of the other two terms. Thus in some embodiments not otherwise explicitly recited, any instance of “comprising” may be replaced by “consisting of” or, alternatively, by “consisting essentially of”, and thus, for the purposes of claim support and construction for “consisting of” format claims, such replacements operate to create yet other alternative embodiments “consisting essentially of” only the elements recited in the original “comprising” embodiment to the exclusion of all other elements.

Moreover, any claim limitation phrased in functional limitation terms covered by 35 USC § 112(6) (post AIA 112(f)) which has a preamble invoking the closed terms “consisting of,” or “consisting essentially of,” should be understood to mean that the corresponding structure(s) disclosed herein define the exact metes and bounds of what the so claimed invention embodiment(s) consists of, or consisting essentially of, to the exclusion of any other elements which do not materially affect the intended purpose of the so claimed embodiment(s).

Devices or system modules that are in at least general communication with each other need not be in continuous communication with each other, unless expressly specified otherwise. In addition, devices or system modules that are in at least general communication with each other may communicate directly or indirectly through one or more intermediaries. Moreover, it is understood that any system components described or named in any embodiment or claimed herein may be grouped or sub-grouped (and accordingly implicitly renamed) in any combination or sub-combination as those skilled in the art can imagine as suitable for the particular application, and still be within the scope and spirit of the claimed embodiments of the present invention. For an example of what this means, if the invention was a controller of a motor and a valve and the embodiments and claims articulated those components as being separately grouped and connected, applying the foregoing would mean that such an invention and claims would also implicitly cover the valve being grouped inside the motor and the controller being a remote controller with no direct physical connection to the motor or internalized valve, as such the claimed invention is contemplated to cover all ways of grouping and/or adding of intermediate components or systems that still substantially achieve the intended result of the invention.

A description of an embodiment with several components in communication with each other does not imply that all such components are required. On the contrary a variety of optional components are described to illustrate the wide variety of possible embodiments of the present invention.

As is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

In the following description and claims, the terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

A “computer” may refer to one or more apparatus and/or one or more systems that are capable of accepting a structured input, processing the structured input according to prescribed rules, and producing results of the processing as output. Examples of a computer may include: a computer; a stationary and/or portable computer; a computer having a single processor, multiple processors, or multi-core processors, which may operate in parallel and/or not in parallel; a general purpose computer; a supercomputer; a mainframe; a super mini-computer; a mini-computer; a workstation; a micro-computer; a server; a client; an interactive television; a web appliance; a telecommunications device with internet access; a hybrid combination of a computer and an interactive television; a portable computer; a tablet personal computer (PC); a personal digital assistant (PDA); a portable telephone; application-specific hardware to emulate a computer and/or software, such as, for example, a digital signal processor (DSP), a field-programmable gate array (FPGA), an application specific integrated circuit (ASIC), an application specific instruction-set processor (ASIP), a chip, chips, a system on a chip, or a chip set; a data acquisition device; an optical computer; a quantum computer; a biological computer; and generally, an apparatus that may accept data, process data according to one or more stored software programs, generate results, and typically include input, output, storage, arithmetic, logic, and control units.

Those of skill in the art will appreciate that where appropriate, some embodiments of the disclosure may be practiced in network computing environments with many types of computer system configurations, including personal computers, hand-held devices, multi-processor systems, microprocessor-based or programmable consumer electronics, network PCs, minicomputers, mainframe computers, and the like. Where appropriate, embodiments may also be practiced in distributed computing environments where tasks are performed by local and remote processing devices that are linked (either by hardwired links, wireless links, or by a combination thereof) through a communications network. In a distributed computing environment, program modules may be located in both local and remote memory storage devices.

“Software” may refer to prescribed rules to operate a computer. Examples of software may include: code segments in one or more computer-readable languages; graphical and or/textual instructions; applets; pre-compiled code; interpreted code; compiled code; and computer programs.

The example embodiments described herein can be implemented in an operating environment comprising computer-executable instructions (e.g., software) installed on a computer, in hardware, or in a combination of software and hardware. The computer-executable instructions can be written in a computer programming language or can be embodied in firmware logic. If written in a programming language conforming to a recognized standard, such instructions can be executed on a variety of hardware platforms and for interfaces to a variety of operating systems. Although not limited thereto, computer software program code for carrying out operations for aspects of the present invention can be written in any combination of one or more suitable programming languages, including an object oriented programming languages and/or conventional procedural programming languages, and/or programming languages such as, for example, Hyper text Markup Language (HTML), Dynamic HTML, Extensible Markup Language (XML), Extensible Stylesheet Language (XSL), Document Style Semantics and Specification Language (DSSSL), Cascading Style Sheets (CSS), Synchronized Multimedia Integration Language (SMIL), Wireless Markup Language (WML), Java™, Jini™, C, C++, Smalltalk, Perl, UNIX Shell, Visual Basic or Visual Basic Script, Virtual Reality Markup Language (VRML), ColdFusion™ or other compilers, assemblers, interpreters or other computer languages or platforms.

Computer program code for carrying out operations for aspects of the present invention may be written in any combination of one or more programming languages, including an object-oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

A network is a collection of links and nodes (e.g., multiple computers and/or other devices connected together) arranged so that information may be passed from one part of the network to another over multiple links and through various nodes. Examples of networks include the Internet, the public switched telephone network, the global Telex network, computer networks (e.g., an intranet, an extranet, a local-area network, or a wide-area network), wired networks, and wireless networks.

The Internet is a worldwide network of computers and computer networks arranged to allow the easy and robust exchange of information between computer users. Hundreds of millions of people around the world have access to computers connected to the Internet via Internet Service Providers (ISPs). Content providers (e.g., website owners or operators) place multimedia information (e.g., text, graphics, audio, video, animation, and other forms of data) at specific locations on the Internet referred to as webpages. Websites comprise a collection of connected, or otherwise related, webpages. The combination of all the websites and their corresponding webpages on the Internet is generally known as the World Wide Web (WWW) or simply the Web.

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

The flowchart and block diagrams in the figures illustrate the architecture, functionality, and operation of possible implementations of systems, methods and computer program products according to various embodiments. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

Further, although process steps, method steps, algorithms or the like may be described in a sequential order, such processes, methods and algorithms may be configured to work in alternate orders. In other words, any sequence or order of steps that may be described does not necessarily indicate a requirement that the steps be performed in that order. The steps of processes described herein may be performed in any order practical. Further, some steps may be performed simultaneously.

It will be readily apparent that the various methods and algorithms described herein may be implemented by, e.g., appropriately programmed general purpose computers and computing devices. Typically, a processor (e.g., a microprocessor) will receive instructions from a memory or like device, and execute those instructions, thereby performing a process defined by those instructions. Further, programs that implement such methods and algorithms may be stored and transmitted using a variety of known media.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article.

The functionality and/or the features of a device may be alternatively embodied by one or more other devices which are not explicitly described as having such functionality/features. Thus, other embodiments of the present invention need not include the device itself.

The term “computer-readable medium” as used herein refers to any medium that participates in providing data (e.g., instructions) which may be read by a computer, a processor or a like device. Such a medium may take many forms, including but not limited to, non-volatile media, volatile media, and transmission media. Non-volatile media include, for example, optical or magnetic disks and other persistent memory. Volatile media include dynamic random access memory (DRAM), which typically constitutes the main memory. Transmission media include coaxial cables, copper wire and fiber optics, including the wires that comprise a system bus coupled to the processor. Transmission media may include or convey acoustic waves, light waves and electromagnetic emissions, such as those generated during radio frequency (RF) and infrared (IR) data communications. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, removable media, flash memory, a “memory stick”, a NFC (near field communication) chip, or any other memory chip or cartridge, a carrier wave as described hereinafter, a cloud based memory service, or any other medium from which a computer can read.

Various forms of computer readable media may be involved in carrying sequences of instructions to a processor. For example, sequences of instruction (i) may be delivered from RAM to a processor, (ii) may be carried over a wireless transmission medium, and/or (iii) may be formatted according to numerous formats, standards or protocols, such as Bluetooth, TDMA, CDMA, 3G.

Where databases are described, it will be understood by one of ordinary skill in the art that (i) alternative database structures to those described may be readily employed, (ii) other memory structures besides databases may be readily employed. Any schematic illustrations and accompanying descriptions of any sample databases presented herein are exemplary arrangements for stored representations of information. Any number of other arrangements may be employed besides those suggested by the tables shown. Similarly, any illustrated entries of the databases represent exemplary information only; those skilled in the art will understand that the number and content of the entries can be different from those illustrated herein. Further, despite any depiction of the databases as tables, an object-based model could be used to store and manipulate the data types of the present invention and likewise, object methods or behaviors can be used to implement the processes of the present invention.

A “computer system” may refer to a system having one or more computers, where each computer may include a computer-readable medium embodying software to operate the computer or one or more of its components. Examples of a computer system may include: a distributed computer system for processing information via computer systems linked by a network; two or more computer systems connected together via a network for transmitting and/or receiving information between the computer systems; a computer system including two or more processors within a single computer; and one or more apparatuses and/or one or more systems that may accept data, may process data in accordance with one or more stored software programs, may generate results, and typically may include input, output, storage, arithmetic, logic, and control units.

A “network” may refer to a number of computers and associated devices that may be connected by communication facilities. A network may involve permanent connections such as cables or temporary connections such as those made through telephone or other communication links. A network may further include hard-wired connections (e.g., coaxial cable, twisted pair, optical fiber, waveguides, etc.) and/or wireless connections (e.g., radio frequency waveforms, free-space optical waveforms, acoustic waveforms, etc.). Examples of a network may include: an internet, such as the Internet; an intranet; a local area network (LAN); a wide area network (WAN); and a combination of networks, such as an internet and an intranet.

As used herein, the “client-side” application should be broadly construed to refer to an application, a page associated with that application, or some other resource or function invoked by a client-side request to the application. A “browser” as used herein is not intended to refer to any specific browser (e.g., Internet Explorer, Safari, FireFox, or the like), but should be broadly construed to refer to any client-side rendering engine that can access and display Internet-accessible resources. A “rich” client typically refers to a non-HTTP based client-side application, such as an SSH or CFIS client. Further, while typically the client-server interactions occur using HTTP, this is not a limitation either. The client server interaction may be formatted to conform to the Simple Object Access Protocol (SOAP) and travel over HTTP (over the public Internet), FTP, or any other reliable transport mechanism (such as IBM® MQSeries® technologies and CORBA, for transport over an enterprise intranet) may be used. Any application or functionality described herein may be implemented as native code, by providing hooks into another application, by facilitating use of the mechanism as a plug-in, by linking to the mechanism, and the like.

Exemplary networks may operate with any of a number of protocols, such as Internet protocol (IP), asynchronous transfer mode (ATM), and/or synchronous optical network (SONET), user datagram protocol (UDP), IEEE 802.x, etc.

Embodiments of the present invention may include apparatuses for performing the operations disclosed herein. An apparatus may be specially constructed for the desired purposes, or it may comprise a general-purpose device selectively activated or reconfigured by a program stored in the device.

Embodiments of the invention may also be implemented in one or a combination of hardware, firmware, and software. They may be implemented as instructions stored on a machine-readable medium, which may be read and executed by a computing platform to perform the operations described herein.

More specifically, as will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

In the following description and claims, the terms “computer program medium” and “computer readable medium” may be used to generally refer to media such as, but not limited to, removable storage drives, a hard disk installed in hard disk drive, and the like. These computer program products may provide software to a computer system. Embodiments of the invention may be directed to such computer program products.

An algorithm is here, and generally, considered to be a self-consistent sequence of acts or operations leading to a desired result. These include physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers or the like. It should be understood, however, that all of these and similar terms are to be associated with the appropriate physical quantities and are merely convenient labels applied to these quantities.

Unless specifically stated otherwise, and as may be apparent from the following description and claims, it should be appreciated that throughout the specification descriptions utilizing terms such as “processing,” “computing,” “calculating,” “determining,” or the like, refer to the action and/or processes of a computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

Additionally, the phrase “configured to” or “operable for” can include generic structure (e.g., generic circuitry) that is manipulated by software and/or firmware (e.g., an FPGA or a general-purpose processor executing software) to operate in a manner that is capable of performing the task(s) at issue. “Configured to” may also include adapting a manufacturing process (e.g., a semiconductor fabrication facility) to fabricate devices (e.g., integrated circuits) that are adapted to implement or perform one or more tasks.

In a similar manner, the term “processor” may refer to any device or portion of a device that processes electronic data from registers and/or memory to transform that electronic data into other electronic data that may be stored in registers and/or memory. A “computing platform” may comprise one or more processors.

Embodiments within the scope of the present disclosure may also include tangible and/or non-transitory computer-readable storage media for carrying or having computer-executable instructions or data structures stored thereon. Such non-transitory computer-readable storage media can be any available media that can be accessed by a general purpose or special purpose computer, including the functional design of any special purpose processor as discussed above. By way of example, and not limitation, such non-transitory computer-readable media can include RAM, ROM, EEPROM, CD-ROM or other optical disk storage, magnetic disk storage or other magnetic storage devices, web based and or cloud based storage, or any other medium which can be used to carry or store desired program code means in the form of computer-executable instructions, data structures, or processor chip design. When information is transferred or provided over a network or another communications connection (either hardwired, wireless, or combination thereof) to a computer, the computer properly views the connection as a computer-readable medium. Thus, any such connection is properly termed a computer-readable medium. Combinations of the above should also be included within the scope of the computer-readable media.

While a non-transitory computer readable medium includes, but is not limited to, a hard drive, compact disc, flash memory, volatile memory, random access memory, magnetic memory, optical memory, web and cloud based memory, semiconductor based memory, phase change memory, optical memory, periodically refreshed memory, and the like; the non-transitory computer readable medium, however, does not include a pure transitory signal per se; i.e., where the medium itself is transitory.

It is to be understood that any exact measurements/dimensions or particular construction materials indicated herein are solely provided as examples of suitable configurations and are not intended to be limiting in any way. Depending on the needs of the particular application, those skilled in the art will readily recognize, in light of the following teachings, a multiplicity of suitable alternative implementation details.

An embodiment of the present invention may provide improved systems and methods for evaluating a medical situation for a patient and improve medical data analysis. A system and method may include an improved interface to take medical information from a user in an agile environment. A graphical user interface may entice users to participate by using objects including but not limited to icons, imagery in general, limited text, audio, and/or video. Using simplified text and imagery to speed up medical history taking may produce significant data for patients, health care workers and medical data analytics. In some embodiments, the graphical user interface (GUI) for taking medical history is a form of user interface that may allow users to interact with electronic devices through graphical icons and visual indicators, in addition to text-based user interfaces, typed command labels and/or text navigation. The taking of medical history in the inventive GUI is usually performed through direct manipulation of the graphical elements. The visual composition and temporal behavior design of the GUI is an important part of the software application programming in the area of medical history taking using human-computer interaction. The goal of the medical history documentation is to enhance the efficiency and usability for the underlying logical design of a stored medical history. Methods of user-centered design for medical history documentation are used to ensure that the visual language introduced in the GUI is well-tailored to the tasks. A system and method may include using a data driven method of filtering and structuring questions to maximize a number of valuable medical questions answered by patients for doctor's visits. Questions are structured to elucidate symptoms and details on symptoms, like timing specifics, provoking or alleviating factors but not limited to these specifics. The questions are structured in an anatomical and or functional fashion, to be able to describe any complaint or symptom. Thereby focus is on the person questioned, and not working according to the scripted interview of medical anamneses, where one might ask questions to rule in or rule out pulmonary emboli which one could describe as disease focused. Systems and methods may collect and data mine then present gathered patient profiles, diagnosis, and outcomes which may be used to make recommendations to patients.

Patients themselves may provide their medical history in an agile environment where unlike common questions in text and standard pull down menus, these questions and answers may be performed by clicking icons, using pull down icon menus and/or audio/video assisted questions. An agile and/or multilingual interface may enable questions and answers to be given irrespective of background and/or language. A method may provide an option to switch between languages (fill in Spanish but conclusion in English for example). Quantification of the process may be used in data analysis algorithms to analyze a patient. For example, if icons are ticked in a medical history taking application, the prediction likelihood of coronary disease is 1% given the known data of previous questionnaires' statistics. Similar or comparable patterns of previous people that used the medical history taking process can be used to compare with. This can be in relation to additional exams performed, diagnostics, prognostics, pharmacotherapeutics, risk and others. For example, with this pattern of answers, in rare cases (1-5%%) pulmonary emboli were detected previously. This is based on for example 100 previous comparable cases. A list of diseases of these for example 100 cases can be given. For example, we do not jump from a smart and small set of questions to a conclusion as “thuisarts.nl” does, or Idana.com, but compare a dataset from an individual, to datasets from previous. This can be a dataset from questionnaire as such, and or enriched with other data from health sensors and other data.

In another embodiment, a medical history taking process may be completed with data on patient previous history, and co-morbidities and situations like smoking or drug abuse and social/psychological context. Many questions may be filled in using icons or other graphical methods. Questions may be asked in relation to body systems/body parts, but also questions related to topics including but not limited to depression, mood, loss of appetite, loss of initiative. Validated questionnaires may be hidden over categories.

Categories of data, unlike text but like discrete data fields may be obtained. Categorized data may be linked to findings from disease later. This may help visualize relations between symptoms and findings. Questions may be placed in categories including but not limited to complaint, character, time, provoking factors, where categories may further more be ordered with a standard stepwise approach. This approach may encompass a qualitative ordering of complaints/aspects in an order of comparability or put in a separate field of not comparable at all. Next it may be validated when a model provides a quantitative basis for this ordering because, later on additional data may be available to enhance a model. Algorithms may clear up details on complaints, expectations, fears, secretions like stools, or urination of secretions from wounds through follow up questions. This may be done by having algorithms focusing on a patient's feedback and not disease related data. For example, fatigue and shortness of breath and chest pain on exertion may be put closer to each other in this ranking than pain in the knee, which is placed in a separate unrelated field. Neck and spine problems may be related to knee/feet problems and neck/spine trouble may also be related to headache trouble. Knee trouble however may not be related to headache.

An order of relatedness may be made within, but also between categories. For example, if sensations of chest pain may be described as burning, tearing, gnawing, sharp, very localized, bursting, excruciating or colicky, the order might be: 1) bursting, 2) sharp, 3) colicky, 4) gnawing, 5) burning, 6) very localized. Excruciating may not be put in this order as this relates to severity more than a sensation of pain and seen as a separate value. Regarding a category of time of complaint, this should be categorized different for different complaints. It is however a very valuable contributor in making a diagnosis. It is very well possible that within a category mainly separate issues may be described, or that arbitrary cutoffs are needed. For example, chest pain on exertion left untreated may be unlikely to be of coronary atherosclerosis (a progressive disease) if it is existing over 10 years, 2 years, 1 year, or 1 month. For flu, complaints for a month may make flu a lot less likely a diagnosis. Moreover, a year of flu may be impossible, so “common sense” ordering may be implemented to prevent big data failures. Timing characteristics may only be relevant within questionnaires of complaints, but not between categories. For example, seven days symptoms might point to flu, more than chronic obstructive pulmonary disease in coughing, but may not differentiate between knee and pulmonary problems. In this way, certain relations may not be made due to limitations entered in the model, others may. Seven days duration may not discriminate a leg from head trouble. Location of pain may and certain patterns in questionnaires may be compared, and others may not be comparable. These timing details of complaints can be used to compare groups with same complaints and different timing intervals, to elucidate relation to diagnoses in a quantitative fashion. Statistics can be added, confidence intervals, rare but relevant serious diseases can be found in differential diagnosis (=list of possible diseases) earlier. Required diagnostics can be focused better, more or less, with large impact on care.

This can be used for research as timing data are largely unknown in medical history taking now.

If data are in a model of symptoms related to eventual diagnosis, a reshuffling of ordering may be needed. The model may enable warning signs when a relation between questions answered and end result is far from an expected result. This may be a way to find common “big data” mistakes and provide more insight to users, to show users what is going on with a model's reasoning. The model may change, according to input suggestions, insights by user (study in website/webApp behavior), development of knowledge, and/or by relating input to diseases discovered later on. Ordering and adjusting and improving the items in a model may not only be done by describing ordering within and between items as separate entities, it too may be done by describing fixed combinations which may characterize a problem much more than the attributing questions. For example, the pattern of complaints physicians now find fitting with cholecystitis, might form a considerable overlap with other disease that physicians now might miss or overlook at first (2% hepatic cancer? 5%, 10% even?). This possible finding that cancer is a relevant disease to consider, might shift the focus of questioning in a classical medical history taking. In our model we ask questions, encompassing questions that cover different aspects, not limiting to the questions one would find fitting for cholecystitis or cancer. Or, one might say, the model finds specifics of complaint, in a total version of the model, all complaints, but “not leading the witness”, not disease centered in its primary structure, but anatomical and or functional oriented, but not limited to. We structured a model of questioning to clarify what a patient's complaints are, and not what to expect in a certain disease.

A model may be using the international standard called Systematized Nomenclature of Medicine “Snomed” with predefined terms, and fulfilling rules and advices to adhere to standards including but not limited to ISO norms, CE and FDA norms, IL7, etc.\

In some embodiments, in a chatbot version or audio or video assisted part as well as graphic/text interface may stimulate patients to put answers in discrete categories as possible to enhance data analyses options. For example, if a free text entree is chosen, a suggestion may be made to re-evaluate existing options and choose the nearest by option. Chatbot or video-based help or online help in general may be forced to get a dataset as categorized as possible.

In some embodiments, a system and method may use graphical depictions of above mentioned “Socrates” questions and others. For example, a time scale may be depicted to give a moment in time and a box with adjustable magnitude for duration. For severity, a scale and smileys may be used. Complaints and other parameters may be depicted by icons. This may facilitate quick ticking of boxes under specific items, not missing items that may be overlooked in a busy health care worker's office. Questions/items each may be labeled in ways including but not limited to a complaint being part of a questionnaire, part of current known medical algorithm, and/or ending up as part of a depression score, a level of protection (can be seen by one doctor one month or any other level), a label for alarm, or a label for specificity (like gynecology is not applicable to men, greenstick fracture not to elderly, or date of start is not a variable linked to a specific disease but only a contributor).

In alternative exemplary embodiment, a graphical questionnaire may be on paper. These may be analyzed by man, or automatically, using advanced scanning technique, or older pattern recognition techniques, like a model with cut out boxes that may be put over the patient paper to get a quick impression if a certain relevant problem is present like infarction. But it may be computerized as well. In other embodiments, the system may comprise of an input device like a computer or smart phone or a pre-made booth in an office. In this system, languages may be alternated easily. Data may be stored locally, transferred to a database, a cloud or electronic patient file. Data alone or with other data of patient or group may be analyzed to develop algorithms, with or without other health care workers, to rule in or rule out disease and suggest other steps. It may generate a written description of a situation to enable interpretation by patient whether it fits the situation the patient may be experiencing well enough.

In some other embodiments, a software module analyzing patient data may be provided. The software module may constitute an automated written report of the data. A written report may be edited by patient and if needed easily translated or adjusted to cultural differences in interpretation of icons (which is minimal). A report may be written and edited orally or written. A written report may resemble a usual medical history and may be used as such, but is now controlled and checked by a patient. With these circumscriptive results of our questionnaires, a large amount of possibilities may arise. In one embodiment, system and methods may focus on auto-diagnosis/prognosis and situations that may either be associated with clear need of medical attention, and those situations that may safely not need medical attention. Treatment and diagnostic algorithms may be suggested.

In some embodiments, data on medical history may be integrated with other data to enable auto-diagnosis and a study of medical history findings in relation to for example CT/echo/lab/electrical/movement/acoustic/echo/visual/sensor and other data. For example, a pattern of answers may be related to renal impairment, most likely infectious of origin, check urine sample. Alternatively, output mode can be selected to physician but also to layperson's answer mode may be selected, then demonstration of a urine sample may be required/provided. Answers to the questionnaire may be provided or not. Answers may be in professional texts or aimed at patients, written or in film/in cartoons or in audio. in additional embodiments, each question/item may be labeled with many labels, like a complaint being part of a questionnaire and/or part of current known medical algorithm, ending up as part of a depression score. For example, like level of protection (may be seen by one doctor one month or any other level), like a label for alarm, or a label for specificity (like gynecology is not applicable to men, greenstick fracture not to elderly, or date of start is not a variable linked to a specific disease but only a contributor). The speed of answer, tone of voice, textual choices, facial expression, pauses in answers, looking away of fearful or other characteristics as variables over time between and within customers in this predictive dataset may be incorporated. To be compared with historic data eventual and within the same patient or customer. For example, if certain data fields are answered in slower fashion, looking away, or other pitch of voice, it is noted and used (if this mode is switched on). Also, within a model of the model, answers can be given by facial expression, motions and or sensing brain activity.

In use, a patient may engage the software with a screen of explanation, options, languages, and/or waivers as interface for history taking. The patient may point to a body part, state it, click in a pull down menu, and/or have an audio and video interaction after which a series of specific scripted follow up questions follow such as, but not limited to, when, where, pain radiating to, severity, etc, in an agile scripted format having icons, cartoons, text/pull down, audio, and/or video according to patients preferences, in which a series of follow up questions is adjusted to a complaint (for example, a script for fainting and a script for chest pain). An option to fill in additional complaints may be provided as a person may have multiple complaints that may not be listed. Output options may be chosen, for patient to adjust/print/mail/read aloud, for health care worker, and for further data analyses. Additional measurements including sensor measurements may be applied. For example, a digital stethoscope applied to the chest wall with instruction if it seemed relevant after the medical history.

In other embodiments, an automated method to get a thorough description of complaints which may be not disease oriented but symptom focused is provided, wherein diagnosis or analyses may be based on relations of symptoms and diseases. Medical history taking may not be best conducted with a medical organ structured approach, but may work better from a patient complaint basis. For example, urinating at night or sleeping up right but not flat may be related to cardiac history taking for a doctor. The above conditions may be a part of problems related to urinating and problems related to sleeping. For statistics and later judgments, symptoms may be related to historic outcomes. Common artificial intelligence problems may be prevented by looking at patterns of answering and/or psychological complaints. Thereby, the invention may be a step back from lean quick ways of including/excluding diseases, getting a thorough dataset in order to have a sound and reliable outcome and transparency to users.

In some embodiments, complaints and/or body secretions may be followed-up in an approach to get all details out in a specific manner with a well described set of follow up questions for each kind of complaint and/or secretion. It may be used separately, for example in a pre-op eye surgery not all body systems may need to be checked so a model may be confined to symptoms related to an upper body like chest pain and/or short of breath. A patient may be discharged with a conclusion and/or presumption that he or she is okay. For a follow up, this invention may check if symptoms have changed without prejudice, checking complaints not directed to a specific disease, state, or complication.

In additional embodiments, a medical history taking process may be enhanced with additional interfacing objects including but not limited to a speedometer showing a time to finish a particular path, questions/quiz like statements where patients may be triggered to fill in parts (for example; ‘do you have lung disease?’), a price for completing more parts or providing data excess (like reduction in price), patients may be suggested that it is better to fill in more, giving a bar from 0-100 for filling more data and positively re-enforcing users to complete data entry of a module within an app, get membership accounts for others by entering data fields (like the personnel data that is needed to form a part of EHR), graphics that increase curiosity (like Pokemon Go, a figure might pop up, as reward), app may explain lower chance of missing things if user stays in the app (like “afraid to miss key findings? Fill in for example the neurology and or rheumatology sector.”), and/or add on questions after conclusion are stated when the app needs clarification in certain cases (for example, if a chest pain path has only been filled in 70%, to motivate). Data analytics may monitor performance and dropping out ratio per screen/interface and time per screen to optimize user experience without missing rarities. Feedback on questions and items as well as user interface may be constantly asked. When no longer in a questionnaire, online secured help may be provided according to specifications of a used method.

In further embodiments, questioning may not be narrowed based on presumptions, but broad questioning may continue, stimulating patients to give a broad insight in their disease and thereby deliver a complete dataset reducing the chance of missing rare diseases. For example, if no hand problems were noted, then questions on fingers may not be pursued but instead, questioning may be directed to problems related to fingers, to rule out signs of rheumatic disease. In another example in case of a flu, questions directed to ear problems may be asked in addition to questions directed to flu symptoms since flu and ear problem may occur together.

A multi-step approach to ask specific questions in a model is not to minimize the amount of questions asked, but to optimally use medical history data to minimize need for additional testing and to not miss rarities. So, from the start of a medical history taking, chances to find a systemic or rare disease may be optimized. Complaints of a person (from sleeping related trouble to visit to toilet & from head to toe) may be demonstrate in a film, icons, text, and/or audio and video, so people can click off a symptom or symptom category oriented to quicken the questioning process. Awareness may be raised regarding an interest from head to toe of a patient. Alternatively, a patient may be asked to click on a static picture of a body where a complaint is located.

The present invention will now be described in detail with reference to embodiments thereof as illustrated in the accompanying drawings.

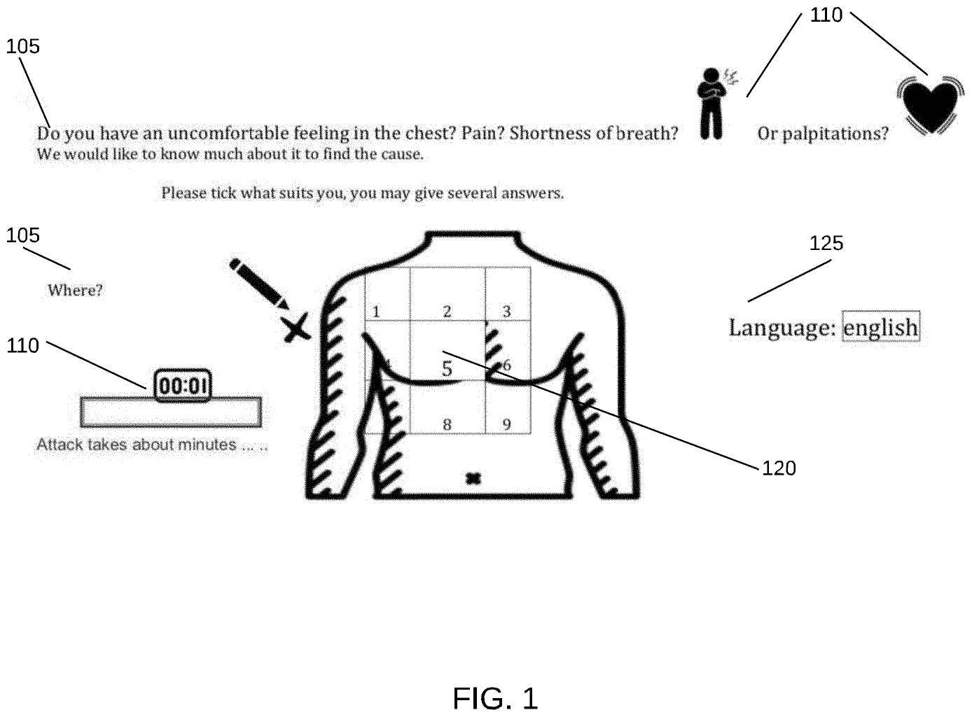

FIG. 1 illustrates an exemplary interface for taking medical history, in accordance with an embodiment of the present invention. An exemplary graphical user interface may enable icon/graphics based selection of area of a specific health problem. Utilizing a graphical interface may entice users to interact and therefore improve data analysis when more users are involved. Utilizing icons and imagery may help ‘gamification’ of a process for taking in medical data from patients, where ‘gamification’ may refer to making the experience of filling out medical history forms more fun for users and or enhance adherence to questions, and or stimulate answering all questions asked. Special consideration may be taken to make sure that key details may be drawn out of patients during a questioning phase. Medical history taking may be further be improved by making sure to emphasize tracking symptoms not just diseases. Tracking symptoms and not diseases may furthermore reduce medical errors by focusing more on known statistics. Furthermore, questions may start off looking for alarming information then later proceed to less relevant information. An exemplary question 105 may be structured for simple straight forward answers which may make responses easily quantifiable. The taking of medical history may then effectively collect compact data which may be utilized to improve data analysis. Furthermore still, a module may insert icons 110 to get concepts across quicker, entice users to participate, and speed up a medical history taking process. Using icons 110 may furthermore reduce a need for excessive text for a questionnaire interface, further speeding up a medical history taking process. Furthermore still, compared to standard long medical forms using icons 110 and simplified text would significantly reduce the time patients spent entering their health information over time. Furthermore still, a graphical user interface may include additional tools to further simplify and improve a quality of resulting data including but not limited to a numeric pad 120 superimposed on a human body, and/or a language selection module 125, and/or various other GUI interfacing/user interfacing tools. Furthermore still, as data quantity may be significantly and/or exponentially increased, data analysis accuracy may improve due to more cases available to train adaptive big data analytics modules. In some embodiments, the graphical user interface (GUI) of this invention is a form of user interface that may allow users to interact with electronic devices through graphical icons and visual indicators, in addition to text-based user interfaces, typed command labels and/or text navigation. The taking of medical history in the inventive GUI is usually performed through direct manipulation of the graphical elements (i.e. icons, imagery). The visual composition and temporal behavior design of the GUI is an important part of the software application programming in the area of medical history taking using human-computer interaction. The goal of the medical history documentation is to enhance the efficiency and usability for the underlying logical design of a stored medical history. Methods of user-centered design for medical history documentation are used to ensure that the visual language introduced in the GUI is well-tailored to the tasks. In an alternative embodiment, medical history data may be added from mobile sensors such as but not limited to wearable activity tracking devices like a FitBit® or similar mobile phone apps or internet of things devices in general. These can be, but are not limited to sensors for pulse, blood pressure, sleeping data, sound, echo, movement, ultrasound, infrared/near infrared, other wavelet frequencies in audio and visual spectrum, electricity of muscle/heart and or brain activity, magnetic resonance imaging, olfactory and laboratory findings, accelerometer and gyroscope data and other data and others. In an alternative embodiment, an interface may include features including but not limited to audio or video reproduction or recording, interfacing with hospital measuring tools, use of minor health care monitoring devices, and/or use of more complicated health measuring processes if the user is trained for them. In an alternative embodiment, an interface may include input by voice, video, hand/body/face gesture, thoughts, neurological input, game controller, mouse, and/or touch.

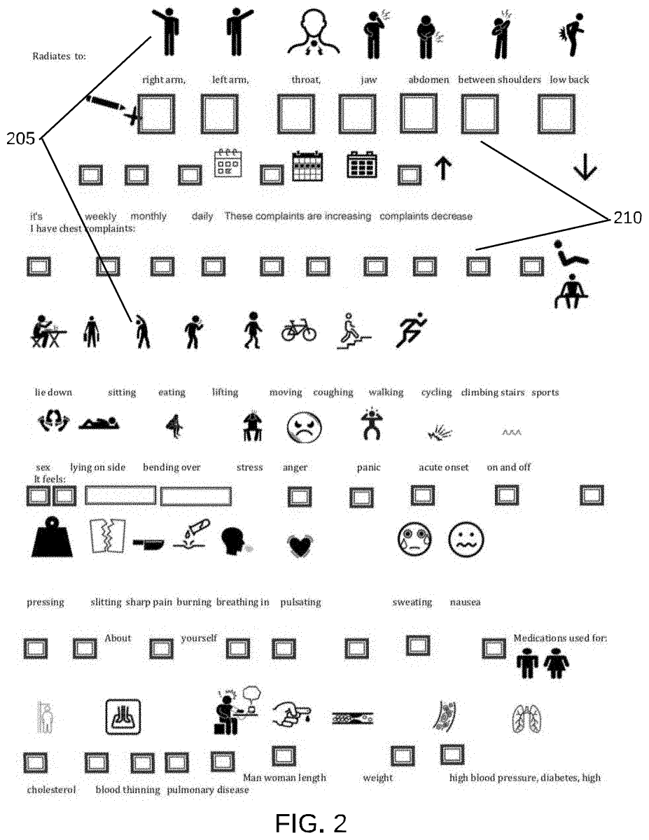

FIG. 2 illustrates exemplary use of icons during interfacing with a patient demonstrating situations in which symptoms occur, in accordance with an embodiment of the present invention. Icons and similarly graphics based information 205 may be provided for a mixture of text and/or icons. Using icons 205/images for questioning and presenting data analysis results may incentivize users to engage in learning about their health and updating their health data regularly. Icons 205 and other graphics may depict patients with various symptoms including but not limited do patients with pain in a certain area of their body or showing signs of symptoms with additional graphics depicting pain or user emotional distress to elicit a response from a user. Using these icons 205 and various graphics user may be able to more quickly make selections for a wider variety of symptoms they may have. By using icons and graphics a questionnaire may be able to cover significantly more ground in shorter time, which in the end would make the resulting data analysis more accurate and help patients more. The use of icon/graphics would currently be a novel way to engage patients in participation of medical data research. Having a user make selections on selection boxes 210 instead of writing out a response would make medical history data more easily quantifiable and therefore improve the overall process. Use of icons 205/graphics may also help to ensure medical terms are being understood correctly by having images to further reinforce a concept. In an alternative embodiment, the graphics may be improved on based on the computing capabilities of the end user device, improvements to the interface may include but are not limited to use of animation, use of current graphics standards, use of visually appealing settings to entice users, and/or use of gaming techniques such as plot and gameplay. In an alternative embodiment, an interface may interact with additional devices such as medical facility tools, sensor technology and/or microphones/speakers.

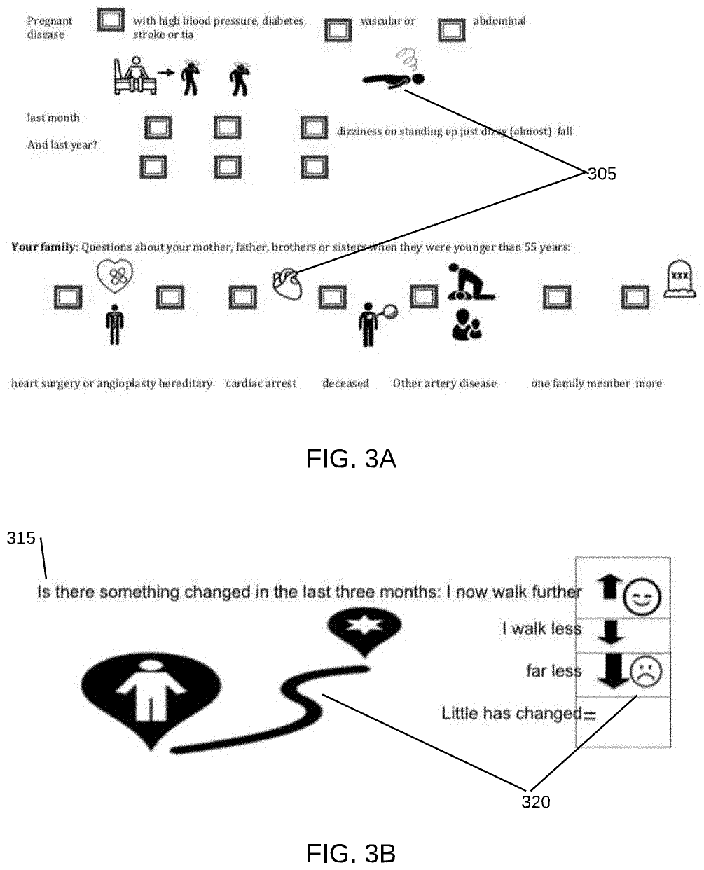

FIGS. 3A & 3B illustrate exemplary questionnaire interfaces, where FIG. 3A illustrates use of icons for questioning a user's health history, where FIG. 3B illustrates use of icons for questioning a user's current health state or condition, in accordance with an embodiment of the invention. A questionnaire interface may be used to extract various types of information from a user. The use of particular icons 305 may help explain a whole scenario/a whole symptom more quickly. A questionnaire may consist of aspects including but not limited to use of simplified questions designed with data quality improvement in consideration. Use of the imagery here shows how much quicker it could be to describe each symptom depicted by an icon 305. A questionnaire may furthermore use icons to further engage users in a medical history questionnaire, use of icons in modern electronics devices is popular and could entices user engagement. In an alternative embodiment, a user may be able to click on an icon to receive more information about symptoms in text or video or audio form. In an alternative embodiment, a user may furthermore be able to see each imagery depicted as a video or animation to have more information to help them make selections. User interface may enable agile use that can be predetermined or selected per item, be it with sound, video, cartoons, icons, text, other languages, or with remote online help. This help can be webchat, audio, video or other method of interaction by lay personnel or health care personnel or friends and or family, for whom a login to the specific item or program is possible. In an alternative embodiment, a user may see the options for a questionnaire in a game setting where a user may be able to enjoy a gaming experience while filling out their medical history questionnaire. This agile gamification approach can be applied by comparing input to others or to one self or to known data. This can be, but not confined to, lists like likelihood of disorders; for example, a relation to a depression scores, a likelihood of presence of ADHD, or other. In an embodiment sliders and or rankings and or depiction of answers and or rewards and or bonus points can be added. This can be in time or over time, for example in relation to adherence to the model. FIG. 3A illustrates an exemplary questionnaire interface, in accordance with an embodiment of the invention. An exemplary interface may be used for a variety of questions 315 where imagery 320 may help convey information more effectively. An interface may use icons and imagery 320 for both a question posed to a user and in the section with response boxes configured to obtain responses. This may help to ensure that a question is more easily quantified and a user's selection is similarly data analysis friendly. Use of icon and imagery may also help promote health, using imagery such as but not limited to happy or sad faces may help reinforce what a user should be doing for their health. In an alternative embodiment, a user may see additional icons or imagery when a selection is made, for example a user may be asked how much more or less they have been walking recently. In an alternative embodiment, a user may then be prompted to input their step tracking device to provide them with more accurate health data. In an alternative embodiment, a user may be able to input a voice recording describing in more detail each of their selections. In an alternative embodiment, a user may be able to speak with a remote medical professional to get more information on the topics they are discussing. In an alternative embodiment the data input of the device combined with sensor data from sensors fitted in the embodiment (so-called medical booth) provide data sets for a remote physician or a computerized algorithm for automated diagnostics and or prognosis and or therapeutics.

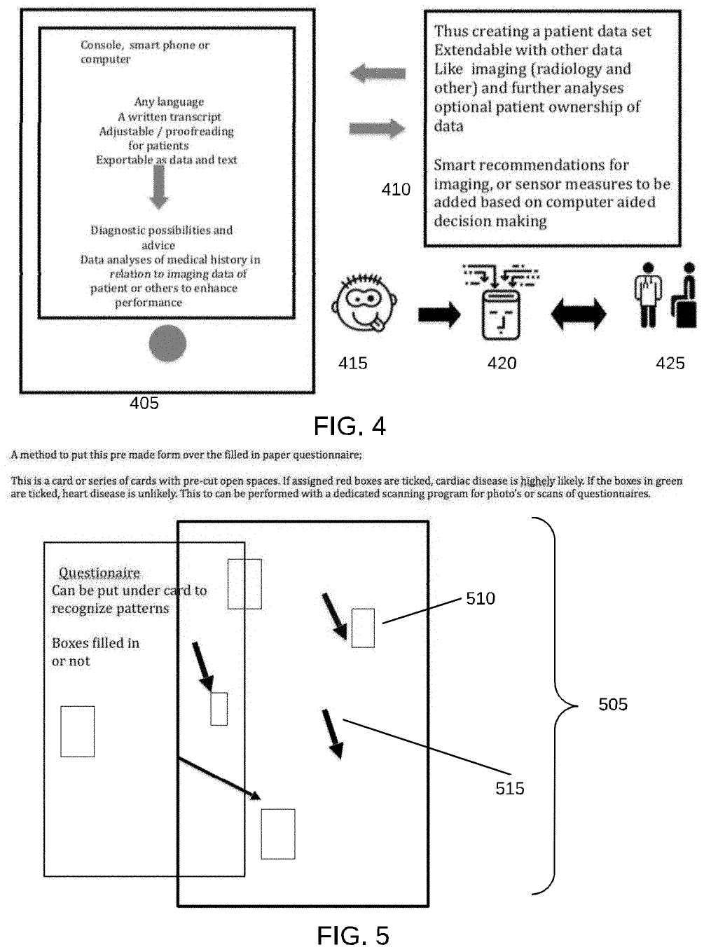

FIG. 4 is an illustration of an overall data processing from an interfacing device to eventual data analysis, in accordance with an embodiment of the present invention. A device 405 may be used to interface with a user in various settings including but not limited to a medical facility, a hospital, a user's home, a location away from their local medical facilities in order to use different medical equipment. A device can be interfaced per item or complete, upon approval, to interact with friend and or family and or other, to guide and or help the one answering questions. The device 405 may be implemented on various types of computers or mobile electronic devices. A user may be able to use a device 405 to input their health data, for example, through the graphical user interface shown in FIGS. 1, 2, 3A and 3B and make additional notes or proofread data input to make sure it is accurate. A user may have a device 405 eventually receive feedback based on the information they provided. Medical history data including but not limited to, medical data and/or responses gathered in the graphical user interface of FIGS. 1, 2, 3A and 3B, may be sent to a data analysis module 410 to provide a high-powered computer to run data analysis. A data analysis module 410 may be located locally or remotely where a more powerful computer may analyze a user's data in comparison to a database. Feedback from a data analysis module 410 may be related to smart medical recommendations, a possible health diagnosis, and/or possible ways to improve their health condition or symptoms. The feedback may be sent and received by the device 405 for review. The overall process for an exemplary patient may then involve use of icons and/or imagery to speedily input their health data 415 such as but not limited to, through the graphical user interface of FIGS. 1, 2, 3A and 3B, health data may be sent to the data analysis module 410 for data analysis 420, then a medical professional may be able to make use of data analysis when seeing their patient 425. In an alternative embodiment, a patient may be able to provide most of the health information autonomously and improved data analysis may allow patients to do more of their healthcare services alone. In an alternative embodiment, a user may immediately be recommended to proceed to a medical measurement tool including but not limited to a hearing exam machine where a user may be able to further perform their medical visit mostly on their own.

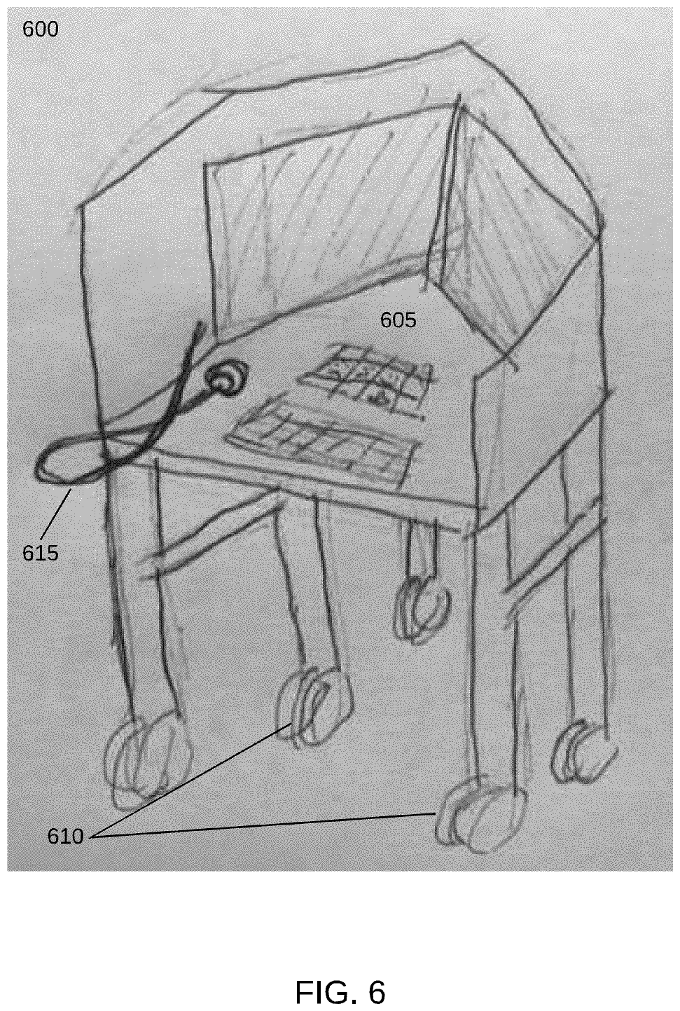

FIG. 5 is an illustration of an exemplary paper-based questionnaire, in accordance with an embodiment of the present invention. A questionnaire may be conveyed to a user if there is no electronic device available or if a situation would call for a less expensive method of inputting health data such as but not limited to a large-scale group of people providing health data. A paper questionnaire 505 may be used in a more effective quicker way when boxes 510 may be selected or punched out so the paper questionnaire 505 may be read/scanned faster by a computer. Use of icons and imagery with a paper questionnaire may still provide a much quicker method of gathering medical information from a user compared to current strictly paper text questions. A paper questionnaire may furthermore be useful for older patients who may consider modern electronic devices confusing, or where electricity is scarce. A paper questionnaire may additionally use arrows 515 in order to guide a user through their answer paths for a certain medical condition. A paper questionnaire 505 may additionally use additional devices to coincide with the questionnaire including but not limited to medical tools. A paper questionnaire may use patterns in order to more quickly have a user fill out their health data. In an alternative embodiment, a paper questionnaire may be electronic paper based. In an alternative embodiment, a paper questionnaire may vary in size and type of input which may include but is not limited to pen/pencil input, hole punch based input, video recording input, and/or voice input.

FIG. 6 illustrates a diagnostic booth, in accordance with an embodiment of the invention. A diagnostic booth 600 may consist of a desk 605 which may be setup in any location and may vary in medical devices 615 available at a particular diagnostic booth. A diagnostic booth 600 may primarily be available to perform both initial pre-screening, regularly scheduled screenings, and/or more advanced post-screening medical procedures or measurements. A diagnostic booth 600 may be mobile 610. In an alternative embodiment, a diagnostic booth 600 may comprise of elements including but not limited to exercise equipment for medical measurements during exercise, image capturing devices for external and internal image capturing, powerful processing on a local computing system for immediate data analysis results for patients, and/or easy to use interfacing options for a user. In an alternative embodiment, a diagnostic booth may be placed at a medical facility to provide a user with significantly more options of medical instruments/tools to use. In an alternative embodiment, a user may be able to have their diagnostic booth medical instruments/tools personalized based on their real time medical history data. Furthermore still, a user may be able to have access to immediate computer driven recommendations for questions to ask a medical professional. Use of a diagnostic booth like this may help to improve any interaction with medical professionals both in person and remotely, give users real-time high-quality information, and provide medical history analysts in general with quality useful data.

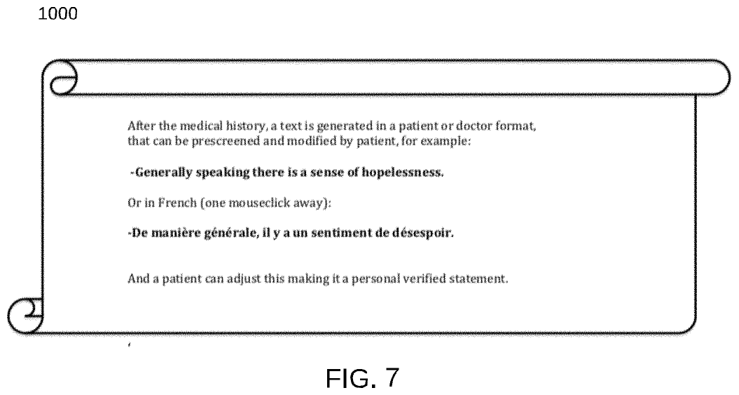

FIG. 7 illustrates an interface providing a presentation of medical history analysis results, in accordance with an embodiment of the invention. An interface 1000 providing results output options may help give patients and medical staff options for how they'd like data results to be output. An interface 1000 providing output options for patients may be located in any module that may provide results from data analysis. This can for example be a number in a depression or heart failure score, constituted from the specific questions that contribute to a specific score or validated score. This can also be for example a listing of answers related to a topic or an overview around a topic or organ or a disease list. In this example a user may have their mood and emotional states tracked by the system, but this may also/or relate to an analysis of physical health. In an alternative embodiment, an interface providing output options for patients may offer options including but not limited to allowing a patient to print out copies of their medical history, print out graphs or visual representations of their health data, visually output data analysis results, and/or audibly output data analysis results. An interface providing output options for patients may help improve data quality by giving patients and medical professionals a most up to date and comprehensive data analysis available. Medical data analysis results output options may allow patients and medical professionals to better give key feedback on ways to improve the system. An interface providing output options for patients may improve medical history taking by giving users more of the information they want in their preferred format which would in turn keep patients and medical professionals engaged in medical data history analysis.

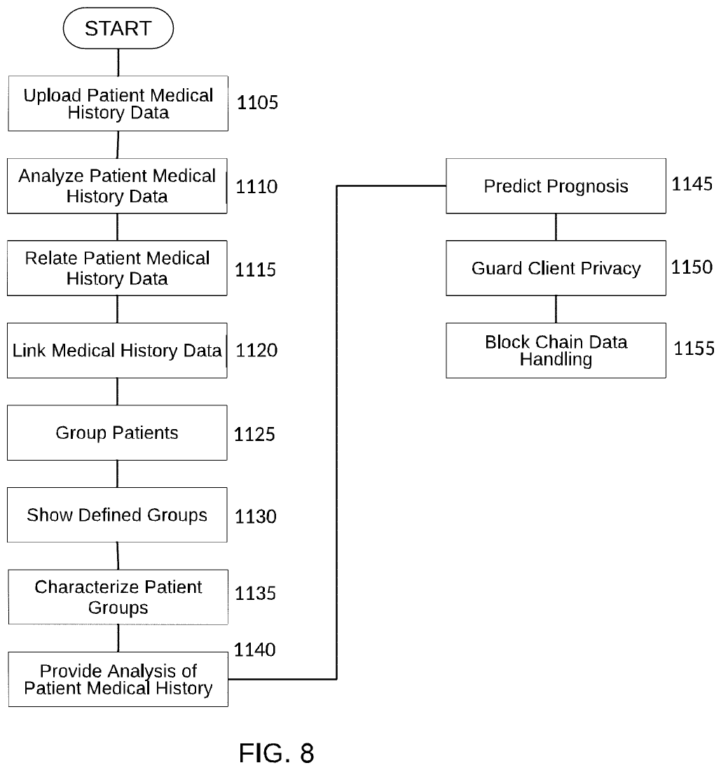

FIG. 8 is a flow chart illustrating a method for performing data analytics on a medical history database, in accordance with en embodiment of the invention. In a Step 1105 a patient's medical history data collected through, but not limited to, the graphical user interface of FIGS. 1, 2, 3A and 3B may be uploaded to a local system such as, but not limited to, a data analysis module 410. This may take place in a location including but not limited to a medical facility, in a pre-screening room, at a patient's home, and/or at a pharmacy. The interface may be located on various devices and/or paper, and an invention may be able to interface with various medical tools including but not limited to ultrasound/radiology/CT/tomography systems, blood pressure readers, and/or heart rate readers. A patient may take safety measures to ensure medical data history privacy including but not limited to requiring authorization for interfacing, reviewing who has viewed their medical history data, ensuring medical facilities are compliant with highly sensitive data security standards such as blockchain based security. In a step 1106 patient (other) data can be uploaded, throughout time, adding diagnostic, prognostic, therapeutic data, electronic health record data, sensor data. In a Step 1110 a patient's medical history data may be analyzed using big data analysis. Big data analysis may include applying learning techniques including but not limited to classification, neural networks, behavior learning, pattern recognition, image/audio/text-based learning, and/or artificial intelligence. The data analysis results may be transmitted and presented to users including but not limited to a device 405, diagnostic booth 600, etc. using additional big data processing including but not limited to filters which may include options for selecting/deselecting results with non-relevant information, date range use, real time updating of each resulting smart recommendation, graph/chart generating, and/or fine-tuning big data analysis. A resulting inventive system may result in easily personalized data searches, options for displaying format, and/or improved accuracy of results after this is regularly being used. In a Step 1115 a patient's medical history data may be related to a patient. A patient may receive results from filter and analysis application. A user may receive related information using means including but not limited to print outs of charts and graphs, print out of text articles related to their conditions, and/or prescriptions. A patient may have their medical history data immediately uploaded to their smart phones where a smart phone may in turn upload recent health tracking sensor data. In a Step 1120 a medical history data may be linked with previous medical history data. Processing related to linking medical history across time may be performed on any device with sufficient processing power, and may be later uploaded to supercomputers where additional processing may be applied to data. In a Step 1125 data for one patient may be grouped by some type of patient group label which may be generated automatically using a data classification module. Groupings may update analysis results and offer additional filters to users who may want to personalize their results. In a Step 1130 defined patient groups for may be presented to a user. Presentation to a user may include but is not limited to paper print outs, displays of various sizes, and/or audibly. In a Step 1135 patient groups may further be characterized by an invention to provide more information. With additional follow up questioning or additional processing an inventive system may provide additional information related to a person's health. Additional information may include but is not limited to smart recommendations, diagnosis, therapy, where to find more information, a checklist of questions to go over with a medical professional, a presentation document downloaded onto their mobile device. In a Step 1140 analysis results of a patient's medical history may be provided to a user. In step 1141 “Mirroring data” the patient answers are related to others who previous filled in the model; how many have filled in comparable answers, in part or total, in which categories, exact or by approach and what is a result in others. Result can be tabelized in relation to specific comparable data, diagnostics used and or result, diagnostics, giving therapeutics used and or prognostic info on the others.

In step 1142 “Personalized profile” the answers of a user can be related to a profile where it is related to others in relations to cardiovascular and or other risk profiles, scores (depression, gravity of disease and or others) and or diagnostics, therapeutics and or prognosis. In step 1143 “Compare personalized profile” user input can be related to others, to other profiles, for example, what in relation to other profiles in relation to sex or age or risk profile (for example, to exemplify effect on prognosis or risk profile if weight is lowered, or medication is taken)

In a Step 1145 a prognosis may be predicted by big data analysis. Prediction may utilize means including but not limited to big data techniques like classification, neural networks, artificial intelligence, and/or computer learning. In a Step 1150 a security module may provide measures to protect a patient's identification information. Security measures may include but are not limited to blockchain use, authorization requirements, encrypted secure network interfacing, and/or fingerprint or image or audio-based recognition. In step 1151 “future research” the patient data can be selected, at a moment, or continuous or multiple moments in part or all and uploaded for coded or un-coded future use in the database of this model or other. Patient data can thus be used for analyses for patient or groups. Patient permission can be entered for data handling, can be regarding the way this is structured (anonymous or not, or under circumstances deblinding), the time of this permission, the amount of data, by whom, and or others. In a Step 1155 an additional security module may be used to improve protection of patients' data, such as but not limited to selective access to medical history records and informing the patient of who was looking at their information and what information. An analysis of a patient's medical history may be performed by a software module on various types of operating systems and/or devices. An analysis of a patient's medical history may help improve data quality by providing significantly more raw data for analysis modules to learn from.

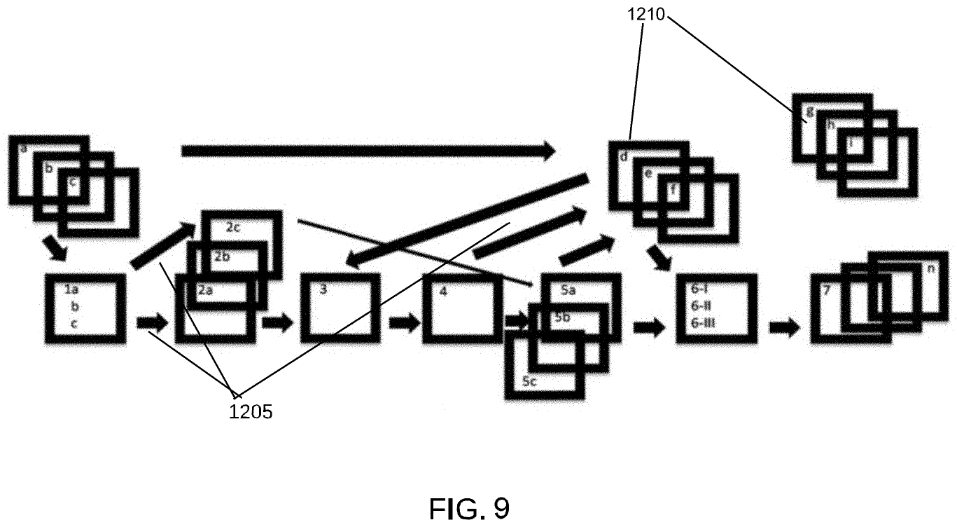

FIG. 9 illustrates a backbone model, in accordance with an embodiment of the present invention. A backbone model for processing medical data may allow a patient to more effectively utilize a system and improve accuracy for a system's data analysis also. A backbone model may provide a way for each medical field to be taken into account. A backbone model for data processing may improve medical history taking by ensuring data may be better quality than current technology. A backbone model for data processing may start with a patient entering general item data including but not limited to identification, general and sometimes needed information, and/or information on services paid for. General information may be depicted in FIG. 9 as lower-case letters. A backbone model may include but is not limited to grouping of medical data 1210 including but not limited to groups such as eye, ear, and/or nose groups. A backbone model for an invention may include but is not limited to a variety of routes/layers 1205. Routes/layers may furthermore use codes for processing. Routes are depicted in FIG. 9 as arrows, and where non Roman numeral numbers denote a length of a route and each route depicted is an example route. There may be plural layers used to add further details about a patient. A layer used may be useful for keeping track of information including but not limited to a time duration for symptoms which may be used retrieve relevant data. Recommended ‘smart routes’ may be suggested based on statistics for other relatively statistically similar patients. For example, a patient with symptoms for several years would have that aspect saved to improve later data analysis and database searching. A backbone model for data processing may help improve data quality by helping to streamline an overall process and recommending common paths. Considering a patient's testing with test results and in the context of the patient information, a short effective path may be recommended. Results for this type of analysis may be output using various graphs and/or charts. A backbone model layer may include but is not limited to a layer for a user's paths through the model. Furthermore, embedded in layers would be an option for when there is a ‘wait and see’ approach taken and a patient may need to keep coming back for re-evaluations. A backbone model layer may include but is not limited to controlling relations and qualifications and variables through routes/paths 1205. A backbone model layer may include but is not limited to application of big data, where smart adaptive computer learning may be used to improve artificial intelligence analysis of medical data. Furthermore, complaints and aspects of a patient may be grouped according to comparability and relatedness. This may be carried out by a data analysis module but adjusted by common sense possessing medical professionals performing data analysis. A backbone model layer may include but is not limited to application of blockchain methods, where security of a system may be up to date with current security standards for large databases. A backbone model layer may include but is not limited to enhanced output of results, where a patient or medical professional may use the system to extract useful information in a personalized manner. A backbone model layer may include but is not limited to selectively storing data, where doing so may increase throughput for passing medical history data around a network or processing modules on a device. A backbone model layer may include but is not limited to database processing for input data, where a database may be searched for selective more relevant information and/or an interfacing user may apply filters in order to personalize results. A backbone model layer may include but is not limited to related financial processing, where secured financial data may indicate which services a patient may have paid extra for. A backbone model layer may include but is not limited to determining results of a diagnostic analysis, where database data may be selectively searched then have that data processed using computer learning techniques. In an alternative embodiment, computer learning techniques may include but are not limited to classification, grouping, artificial intelligence, adaptive learning, neural networks, and/or smart recommendations. A backbone model may include but is not limited to outputting data to medical history records database for future use, where data may be selectively uploaded by patients and a set of data for that interaction may be stored. A backbone model may include but is not limited to post-analysis questioning of patient, where a patient may return to each medical issue and ensure they have all relevant information they need. A backbone model may include but is not limited to additional forms related to legal or sports related surgery information processing, where a user may need to authorize that they searched for medical information, what they searched for, and for what purpose. A backbone model may include but is not limited to providing additional expanding information about topics, where a user may select text or images to get more related information. A backbone model may include but is not limited to background services for the intention's module, where real time medical data processing may continuously update the system to ensure an improvement of quality from the medical history data. Furthermore, a backbone model may be utilized to help a patient have more specific customizable privacy settings. In an alternative embodiment, a backbone model may be split or combined with other models in order to personalize the services to a patient. In an alternative embodiment a backbone model structure itself may be customized for specific conditions, groups of patients, and/or other medical history data statistics.

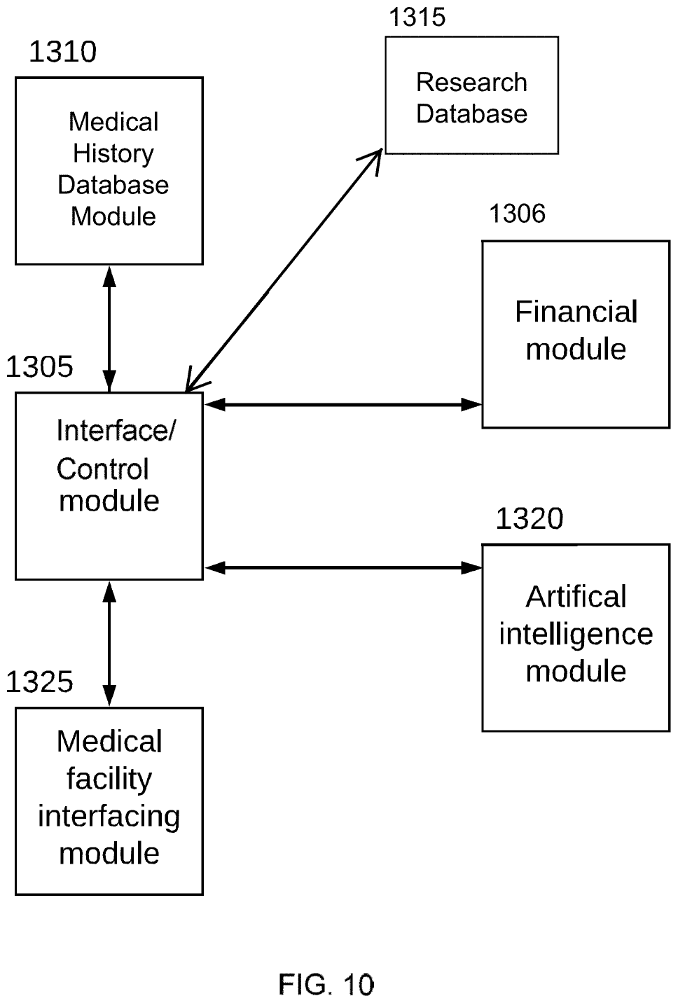

FIG. 10 is a block diagram illustrating a software system modules diagram, in accordance with an embodiment of the invention. An interface/control module 1305 may interface with numerous modules located locally or remotely. An interface/control module 1305 may interface with a medical history database module 1310 which may be used for storing data and performing storage searching on data. Data may be stored both raw and/or as charts and o/r related file types. This data may then be searched through for later machine learning based analysis on the data sets. Furthermore interface/control module 1305 may interface with a financial data module 1306, which may perform secure payment processing and checks on paid for user services. A language model may be used to translate or code information into international standard codes (like SNOMED codes) for output to research database 1315. Furthermore interface/control module 1305 may interface with an artificial intelligence module 1320, which may perform learning techniques on data in forms including but not limited to raw data and/or image data and/or previous data analysis results. Artificial intelligence module 1320 may then perform smart recommendations based on related data analysis performed. Furthermore, interface/control module 1305 may interface with a medical facility interface 1325, which may perform data transferring between a medical facility and a user's interfacing device. The medical facility interface 1325 may perform real time data transferring to make the most of any interaction between a medical facility and a patient

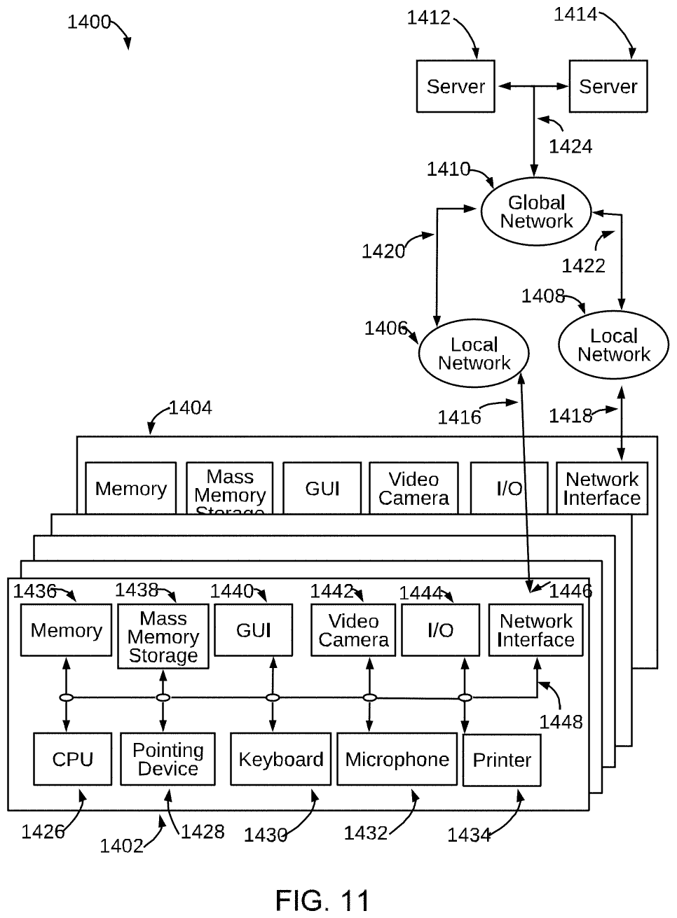

FIG. 11 is a block diagram depicting an exemplary client/server system which may be used by an exemplary web-enabled/networked embodiment of the present invention.

A communication system 1400 includes a multiplicity of clients with a sampling of clients denoted as a client 1402 and a client 1404, a multiplicity of local networks with a sampling of networks denoted as a local network 1406 and a local network 1408, a global network 1410 and a multiplicity of servers with a sampling of servers denoted as a server 1412 and a server 1414.

Client 1402 may communicate bi-directionally with local network 1406 via a communication channel 1416. Client 1404 may communicate bi-directionally with local network 1408 via a communication channel 1418. Local network 1406 may communicate bi-directionally with global network 1410 via a communication channel 1420. Local network 1408 may communicate bi-directionally with global network 1410 via a communication channel 1422. Global network 1410 may communicate bi-directionally with server 1412 and server 1414 via a communication channel 1424. Server 1412 and server 1414 may communicate bi-directionally with each other via communication channel 1424. Furthermore, clients 1402, 1404, local networks 1406, 1408, global network 1410 and servers 1412, 1414 may each communicate bi-directionally with each other.

In one embodiment, global network 1410 may operate as the Internet. It will be understood by those skilled in the art that communication system 1400 may take many different forms. Non-limiting examples of forms for communication system 1400 include local area networks (LANs), wide area networks (WANs), wired telephone networks, wireless networks, or any other network supporting data communication between respective entities.

Clients 1402 and 1404 may take many different forms. Non-limiting examples of clients 1402 and 1404 include personal computers, personal digital assistants (PDAs), cellular phones and smartphones.

Client 1402 includes a CPU 1426, a pointing device 1428, a keyboard 1430, a microphone 1432, a printer 1434, a memory 1436, a mass memory storage 1438, a GUI 1440, a video camera 1442, an input/output interface 1444 and a network interface 1446.

CPU 1426, pointing device 1428, keyboard 1430, microphone 1432, printer 1434, memory 1436, mass memory storage 1438, GUI 1440, video camera 1442, input/output interface 1444 and network interface 1446 may communicate in a unidirectional manner or a bi-directional manner with each other via a communication channel 1448. Communication channel 1448 may be configured as a single communication channel or a multiplicity of communication channels.

CPU 1426 may be comprised of a single processor or multiple processors. CPU 1426 may be of various types including micro-controllers (e.g., with embedded RAM/ROM) and microprocessors such as programmable devices (e.g., RISC or SISC based, or CPLDs and FPGAs) and devices not capable of being programmed such as gate array ASICs (Application Specific Integrated Circuits) or general purpose microprocessors.

As is well known in the art, memory 1436 is used typically to transfer data and instructions to CPU 1426 in a bi-directional manner. Memory 1436, as discussed previously, may include any suitable computer-readable media, intended for data storage, such as those described above excluding any wired or wireless transmissions unless specifically noted. Mass memory storage 1438 may also be coupled bi-directionally to CPU 1426 and provides additional data storage capacity and may include any of the computer-readable media described above. Mass memory storage 1438 may be used to store programs, data and the like and is typically a secondary storage medium such as a hard disk. It will be appreciated that the information retained within mass memory storage 1438, may, in appropriate cases, be incorporated in standard fashion as part of memory 1436 as virtual memory.

CPU 1426 may be coupled to GUI 1440. GUI 1440 enables a user to view the operation of computer operating system and software. CPU 1426 may be coupled to pointing device 1428. Non-limiting examples of pointing device 1428 include computer mouse, trackball and touchpad. Pointing device 1428 enables a user with the capability to maneuver a computer cursor about the viewing area of GUI 1440 and select areas or features in the viewing area of GUI 1440. CPU 1426 may be coupled to keyboard 1430. Keyboard 1430 enables a user with the capability to input alphanumeric textual information to CPU 1426. CPU 1426 may be coupled to microphone 1432. Microphone 1432 enables audio produced by a user to be recorded, processed and communicated by CPU 1426. CPU 1426 may be connected to printer 1434. Printer 1434 enables a user with the capability to print information to a sheet of paper. CPU 1426 may be connected to video camera 1442. Video camera 1442 enables video produced or captured by user to be recorded, processed and communicated by CPU 1426.

CPU 1426 may also be coupled to input/output interface 1444 that connects to one or more input/output devices such as such as CD-ROM, video monitors, track balls, mice, keyboards, microphones, touch-sensitive displays, transducer card readers, magnetic or paper tape readers, tablets, styluses, voice or handwriting recognizers, or other well-known input devices such as, of course, other computers.

Finally, CPU 1426 optionally may be coupled to network interface 1446 which enables communication with an external device such as a database or a computer or telecommunications or internet network using an external connection shown generally as communication channel 1416, which may be implemented as a hardwired or wireless communications link using suitable conventional technologies. With such a connection, CPU 1426 might receive information from the network, or might output information to a network in the course of performing the method steps described in the teachings of the present invention.

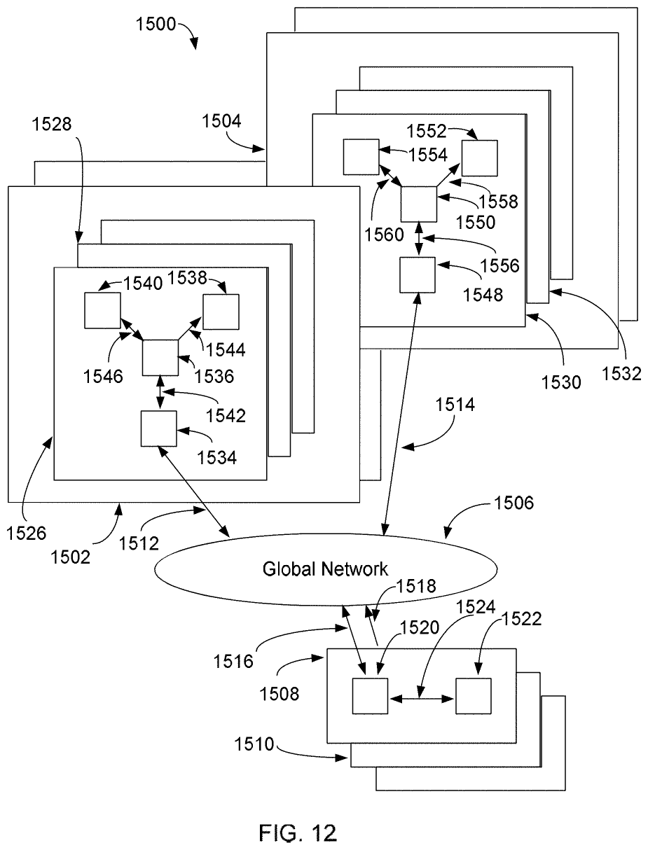

FIG. 12 illustrates a block diagram depicting a conventional client/server communication system, which may be used by an exemplary web-enabled/networked embodiment of the present invention.

A communication system 1500 includes a multiplicity of networked regions with a sampling of regions denoted as a network region 1502 and a network region 1504, a global network 1506 and a multiplicity of servers with a sampling of servers denoted as a server device 1508 and a server device 1510.

Network region 1502 and network region 1504 may operate to represent a network contained within a geographical area or region. Non-limiting examples of representations for the geographical areas for the networked regions may include postal zip codes, telephone area codes, states, counties, cities and countries. Elements within network region 1502 and 1504 may operate to communicate with external elements within other networked regions or within elements contained within the same network region.

In some implementations, global network 1506 may operate as the Internet. It will be understood by those skilled in the art that communication system 1500 may take many different forms. Non-limiting examples of forms for communication system 1500 include local area networks (LANs), wide area networks (WANs), wired telephone networks, cellular telephone networks or any other network supporting data communication between respective entities via hardwired or wireless communication networks. Global network 1506 may operate to transfer information between the various networked elements.

Server device 1508 and server device 1510 may operate to execute software instructions, store information, support database operations and communicate with other networked elements. Non-limiting examples of software and scripting languages which may be executed on server device 1508 and server device 1510 include C, C++, C# and Java.

Network region 1502 may operate to communicate bi-directionally with global network 1506 via a communication channel 1512. Network region 1504 may operate to communicate bi-directionally with global network 1506 via a communication channel 1514. Server device 1508 may operate to communicate bi-directionally with global network 1506 via a communication channel 1516. Server device 1510 may operate to communicate bi-directionally with global network 1506 via a communication channel 1518. Network region 1502 and 1504, global network 1506 and server devices 1508 and 1510 may operate to communicate with each other and with every other networked device located within communication system 1500.

Server device 1508 includes a networking device 1520 and a server 1522. Networking device 1520 may operate to communicate bi-directionally with global network 1506 via communication channel 1516 and with server 1522 via a communication channel 1524. Server 1522 may operate to execute software instructions and store information.

Network region 1502 includes a multiplicity of clients with a sampling denoted as a client 1526 and a client 1528. Client 1526 includes a networking device 1534, a processor 1536, a GUI 1538 and an interface device 1540. Non-limiting examples of devices for GUI 1538 include monitors, televisions, cellular telephones, smartphones and PDAs (Personal Digital Assistants). Non-limiting examples of interface device 1540 include pointing device, mouse, trackball, scanner and printer. Networking device 1534 may communicate bi-directionally with global network 1506 via communication channel 1512 and with processor 1536 via a communication channel 1542. GUI 1538 may receive information from processor 1536 via a communication channel 1544 for presentation to a user for viewing. Interface device 1540 may operate to send control information to processor 1536 and to receive information from processor 1536 via a communication channel 1546. Network region 1504 includes a multiplicity of clients with a sampling denoted as a client 1530 and a client 1532. Client 1530 includes a networking device 1548, a processor 1550, a GUI 1552 and an interface device 1554. Non-limiting examples of devices for GUI 1538 include monitors, televisions, cellular telephones, smartphones and PDAs (Personal Digital Assistants). Non-limiting examples of interface device 1540 include pointing devices, mousse, trackballs, scanners and printers. Networking device 1548 may communicate bi-directionally with global network 1506 via communication channel 1514 and with processor 1550 via a communication channel 1556. GUI 1552 may receive information from processor 1550 via a communication channel 1558 for presentation to a user for viewing. Interface device 1554 may operate to send control information to processor 1550 and to receive information from processor 1550 via a communication channel 1560.

For example, consider the case where a user interfacing with client 1526 may want to execute a networked application. A user may enter the IP (Internet Protocol) address for the networked application using interface device 1540. The IP address information may be communicated to processor 1536 via communication channel 1546. Processor 1536 may then communicate the IP address information to networking device 1534 via communication channel 1542. Networking device 1534 may then communicate the IP address information to global network 1506 via communication channel 1512. Global network 1506 may then communicate the IP address information to networking device 1520 of server device 1508 via communication channel 1516. Networking device 1520 may then communicate the IP address information to server 1522 via communication channel 1524. Server 1522 may receive the IP address information and after processing the IP address information may communicate return information to networking device 1520 via communication channel 1524. Networking device 1520 may communicate the return information to global network 1506 via communication channel 1516. Global network 1506 may communicate the return information to networking device 1534 via communication channel 1512. Networking device 1534 may communicate the return information to processor 1536 via communication channel 1542. Processor 1576 may communicate the return information to GUI 1578 via communication channel 1544. User may then view the return information on GUI 1538 res may be hidden over categories.

Those skilled in the art will readily recognize, in light of and in accordance with the teachings of the present invention, that any of the foregoing steps and/or system modules may be suitably replaced, reordered, removed and additional steps and/or system modules may be inserted depending upon the needs of the particular application, and that the systems of the foregoing embodiments may be implemented using any of a wide variety of suitable processes and system modules, and is not limited to any particular computer hardware, software, middleware, firmware, microcode and the like. For any method steps described in the present application that can be carried out on a computing machine, a typical computer system can, when appropriately configured or designed, serve as a computer system in which those aspects of the invention may be embodied.

Those skilled in the art will readily recognize, in light of and in accordance with the teachings of the present invention, that any of the foregoing steps may be suitably replaced, reordered, removed and additional steps may be inserted depending upon the needs of the particular application. Moreover, the prescribed method steps of the foregoing embodiments may be implemented using any physical and/or hardware system that those skilled in the art will readily know is suitable in light of the foregoing teachings. For any method steps described in the present application that can be carried out on a computing machine, a typical computer system can, when appropriately configured or designed, serve as a computer system in which those aspects of the invention may be embodied. Thus, the present invention is not limited to any particular tangible means of implementation.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

It is noted that according to USA law 35 USC § 112 (1), all claims must be supported by sufficient disclosure in the present patent specification, and any material known to those skilled in the art need not be explicitly disclosed. However, 35 USC § 112 (6) requires that structures corresponding to functional limitations interpreted under 35 USC § 112 (6) must be explicitly disclosed in the patent specification. Moreover, the USPTO's Examination policy of initially treating and searching prior art under the broadest interpretation of a “mean for” or “steps for” claim limitation implies that the broadest initial search on 35 USC § 112(6) (post AIA 112(f)) functional limitation would have to be conducted to support a legally valid Examination on that USPTO policy for broadest interpretation of “mean for” claims. Accordingly, the USPTO will have discovered a multiplicity of prior art documents including disclosure of specific structures and elements which are suitable to act as corresponding structures to satisfy all functional limitations in the below claims that are interpreted under 35 USC § 112(6) (post AIA 112(f)) when such corresponding structures are not explicitly disclosed in the foregoing patent specification. Therefore, for any invention element(s)/structure(s) corresponding to functional claim limitation(s), in the below claims interpreted under 35 USC § 112(6) (post AIA 112(f)), which is/are not explicitly disclosed in the foregoing patent specification, yet do exist in the patent and/or non-patent documents found during the course of USPTO searching, Applicant(s) incorporate all such functionally corresponding structures and related enabling material herein by reference for the purpose of providing explicit structures that implement the functional means claimed. Applicant(s) request(s) that fact finders during any claims construction proceedings and/or examination of patent allowability properly identify and incorporate only the portions of each of these documents discovered during the broadest interpretation search of 35 USC § 112(6) (post AIA 112(f)) limitation, which exist in at least one of the patent and/or non-patent documents found during the course of normal USPTO searching and or supplied to the USPTO during prosecution. Applicant(s) also incorporate by reference the bibliographic citation information to identify all such documents comprising functionally corresponding structures and related enabling material as listed in any PTO Form-892 or likewise any information disclosure statements (IDS) entered into the present patent application by the USPTO or Applicant(s) or any 3^{rd} parties. Applicant(s) also reserve its right to later amend the present application to explicitly include citations to such documents and/or explicitly include the functionally corresponding structures which were incorporate by reference above.

Thus, for any invention element(s)/structure(s) corresponding to functional claim limitation(s), in the below claims, that are interpreted under 35 USC § 112(6) (post AIA 112(f)), which is/are not explicitly disclosed in the foregoing patent specification, Applicant(s) have explicitly prescribed which documents and material to include the otherwise missing disclosure, and have prescribed exactly which portions of such patent and/or non-patent documents should be incorporated by such reference for the purpose of satisfying the disclosure requirements of 35 USC § 112 (6). Applicant(s) note that all the identified documents above which are incorporated by reference to satisfy 35 USC § 112 (6) necessarily have a filing and/or publication date prior to that of the instant application, and thus are valid prior documents to incorporated by reference in the instant application.

Having fully described at least one embodiment of the present invention, other equivalent or alternative methods of implementing medical data collection and analysis according to the present invention will be apparent to those skilled in the art. Various aspects of the invention have been described above by way of illustration, and the specific embodiments disclosed are not intended to limit the invention to the particular forms disclosed. The particular implementation of the medical data collection and analysis may vary depending upon the particular context or application. By way of example, and not limitation, the medical data collection and analysis described in the foregoing were principally directed to improving quality of a patient's medical data and improving the quality of related data analysis implementations; however, similar techniques may instead be applied to big data analysis for non medical information about people, big data analysis for vehicle condition tracking, big analysis for learning related issues, big data analysis for child development, big data analyses for legal maters. For example, our model can fit to ask a series of questions, to describe a legal issue in a systematic fashion, fitting aspects in categories and forming a quantitative approach to legal settings, be it private, criminal, financial or other parts of law. The detailed but questionnaire is set to fit to all aspects of a legal setting, just as the here above description of medical history, aims to describe any medical condition and its specifics. For this, the key complaint in a physical condition, can be compared to a key problem in legal matters, to be specified with extensive list of questions to quantify data. For example; details on divorce can be explored in the model, with extended questions on amount and times related to a will, a fortune, assets, real estate, cars, income, pension claims, inheritance, financial prospects and risks, children, pets and index marriage agreements, following an order as the index of an extensive legal textbook. In the text one could replace the words health/physician/medical with legal terms, like legal, lawyer, etcetera. The database can be formed working from a legal textbook, fitting everything in structured questions including previous jurisdiction and laws if relevant, just as extensive datasets fill our medical questionnaire. Again, not to fit to a solution or previous case, but to systematically describe the setting, in a way that allows quantitative analyses of aspects in the inquiry., which implementations of the present invention are contemplated as within the scope of the present invention. The invention is thus to cover all modifications, equivalents, and alternatives falling within the spirit and scope of the following claims. It is to be further understood that not all of the disclosed embodiments in the foregoing specification will necessarily satisfy or achieve each of the objects, advantages, or improvements described in the foregoing specification.

Claim elements and steps herein may have been numbered and/or lettered solely as an aid in readability and understanding. Any such numbering and lettering in itself is not intended to and should not be taken to indicate the ordering of elements and/or steps in the claims.

The corresponding structures, materials, acts, and equivalents of all means or step plus function elements in the claims below are intended to include any structure, material, or act for performing the function in combination with other claimed elements as specifically claimed.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The Abstract is provided to comply with 37 C.F.R. Section 1.72(b) requiring an abstract that will allow the reader to ascertain the nature and gist of the technical disclosure. That is, the Abstract is provided merely to introduce certain concepts and not to identify any key or essential features of the claimed subject matter. It is submitted with the understanding that it will not be used to limit or interpret the scope or meaning of the claims.

The following claims are hereby incorporated into the detailed description, with each claim standing on its own as a separate embodiment.

What is claimed is:

1. A non-transitory computer-readable storage medium with an executable program stored thereon, wherein the program instructs one or more processors to perform a method, the method comprising the following steps:
presenting a first graphical user interface that comprises a graphics based selection and text based selection for taking in medical history data from user input provided through the first graphical user interface, said graphics based selection is configured to convey various medical conditions using a numeric pad superimposed on a human body, an icon showing a palpitating heart, and an icon depicting a person having a chest or breathing problem;
presenting a second graphical user interface that comprises icons depicting pain or symptom in a certain area of a body and a first selection box for each of the depicted pain or symptom, said icons and said first selection boxes operable for eliciting a response corresponding to a pain or symptom experienced by a user by receiving user input using the second graphical user interface;
tracking the pain or symptom, not diseases, contained in said first selection box;
presenting a third graphical user interface that comprises icons or imagery depicting a medical health history with corresponding response boxes configured to question and obtain response answers regarding said medical health history by receiving user input using the third graphical user interface;
presenting a fourth graphical user interface that comprises icons or imagery configured to depict current health conditions and a second selection box for each of the current health conditions depicted by the icons and imagery to obtain at least one indication regarding said current health condition;
processing the medical history data, the response corresponding to the pain or symptom, the response answers regarding said medical health history, and the indication regarding said current health condition to produce user medical health data; and
presenting the user medical health data on a display device for review by the user.

2. The method of claim 1 further comprising the step of sending said selection from said first graphical user interface, said response corresponding to said depicted pain or symptom from said second graphical user interface, said responses regarding said medical health history from said third graphical user interface, and responses regarding said current health condition from said fourth graphical user interface to a data analysis module for medical data analysis.

3. The method of claim 2 further comprising the step of receiving a feedback from said data analysis module, said feedback comprises at least one of, a medical recommendation, a possible health diagnosis, and ways to improve a health condition or symptom.

4. The method of claim 1 further comprising the step of inserting at least one icon into a predefined portion of said first graphical user interface to generally speed up said medical history taking process.

5. The method of claim 4, in which said second graphical user interface further comprises selection boxes instead of writing out a response, said selection boxes are configured to be operable for making medical history data substantially quantifiable.

6. The method of claim 5, further comprising the step of presenting a questionnaire interface that is configured to extract additional medical information.

7. The method of claim 2 further comprising the step of receiving at least a medical data analysis result, wherein said analysis comprises an application of at least one of, a neural network, a pattern recognition, and artificial intelligence technique.

8. The method of claim 7, in which said medical data analysis result include a boxed selection for selecting or deselecting results with non-relevant information, a date range use, a real time updating of each resulting medical recommendation, a graph or chart generator, or a fine tuning of the medical data analysis.

9. The method of claim 8, in which said medical data analysis result further include medical history data linked with previous medical history data.

10. The method of claim 9, in which said medical data analysis result is a medical data analysis result of a patient belonging to a predefined patient grouping.

11. A system comprising:
a mobile device, said mobile device comprises an executable software program that is configured to perform a method of documenting a medical history of a patient;
in which the mobile device is operable to present a graphics and text based selection for taking in medical history data, said graphics based selection is configured to convey various medical conditions, in which said graphics based selection comprises at least one of, a numeric pad superimposed on a human body, an icon showing a palpitating heart, an icon depicting a person having a chest or breathing problem;
in which the mobile device is operable to present icons depicting pain or symptom in a certain area of a body and response boxes configured to elicit a response corresponding to said depicted pain or symptom;
in which the mobile device is operable to present icons and imagery configured to question and obtain responses regarding a health history;
in which the mobile device is operable to present icons and imagery configured to question and obtain responses regarding a current health condition; and
wherein the mobile device further comprises a data analysis module, said data analysis module comprises a computer to run data analysis on said selection from said first graphical user interface, said response corresponding to said depicted pain or symptom from said second graphical user interface, responses regarding said health history from said third graphical user interface, and responses regarding said current health condition from said fourth graphical user interface to a data analysis module for medical data analysis.

12. The system of claim 11 in which the mobile device is further operable to present at least a paper questionnaire for conveying to a user if there is no electronic device available or if a situation would call for a less expensive method of inputting health data, said paper questionnaire comprises boxes configured to be selected or punched out so the paper questionnaire is read or scanned by a computer.

13. The system of claim 12, further comprising a diagnostic booth, wherein said diagnostic booth is configured to be operable for at least one of, an initial pre-screening, a regularly scheduled screenings, and advanced post-screening medical procedures or measurements.

14. The system of claim 13, in which said diagnostic booth comprises at least one of, an exercise equipment for medical measurements during exercise, an image capturing device for external and internal image capturing, a computing system for providing immediate data analysis results for patients, and a device running a graphical user interface for taking in medical history data.

15. A non-transitory computer-readable storage medium with an executable program stored thereon, wherein the program instructs one or more processors to perform a method, the method comprising the following steps:
presenting a first graphical user interface that comprises a graphics based selection and text based selection for taking in medical history data, said graphics based selection is configured to convey various medical conditions from user input provided through the first graphical user interface using a numeric pad superimposed on a human body, an icon showing a palpitating heart, and an icon depicting a person having a chest or breathing problem;
presenting a second graphical user interface that comprises icons depicting pain or symptom in a certain area of a body and a first selection box for each of the depicted pain or symptom, said icons and said first selection boxes being operable for eliciting a response corresponding to a pain or symptom experienced by a user by receiving user input using the second graphical user interface;
tracking the pain or symptom, not diseases, contained in said first selection box;
presenting a third graphical user interface that comprises icons or imagery depicting a medical health history with corresponding response boxes configured to question and obtain response answers regarding said medical health history by receiving user input using the third graphical user interface;
presenting a fourth graphical user interface that comprises icons or imagery configured to depict current health conditions and a second selection box for each of the current health conditions depicted by the icons and imagery to obtain at least one indication regarding said current health condition; and
sending said selection from said first graphical user interface, said response corresponding to said depicted pain or symptom from said second graphical user interface, responses regarding said health history from said third graphical user interface, and responses regarding said current health condition from said fourth graphical user interface to a data analysis module for medical data analysis.

16. The method of claim 15 further comprising the step of receiving a feedback from said data analysis module, said feedback comprises at least one of, a medical recommendation, a possible health diagnosis, and ways to improve a health condition or symptom.

17. The method of claim 16 further comprising the step of inserting icons into portions of said first graphical user interface to generally speed up said medical history taking process.

18. The method of claim 16, in which said second graphical user interface in which the first selection boxes instead of writing out a response, said first selection boxes are operable for making a medical history data substantially quantifiable and improve the overall process.

19. The method of claim 15 further comprising the step of receiving at least a medical data analysis result, wherein said analysis comprises an application of at least one of, a neural network, a pattern recognition, and an artificial intelligence technique.

20. The method of claim 19, in which said medical data analysis result include a medical history data linked with a previous medical history data, and in which said medical data analysis result further include at least a third selection box for selecting or deselecting results with non-relevant information, date range use, real time updating of each resulting medical recommendation, graph or chart generating, or fine tuning of the medical data analysis.
